(19) United States
(12) Patent Application Publication
(10) Pub. No.: US 2026/0072039 A1
(43) Pub. Date: Mar. 12, 2026

(54) PREVENTION AND TREATMENT OF CYTOKINE RELEASE SYNDROME AND NEUROTOXICITY ASSOCIATED WITH CAR-T CELL THERAPY

(71) Applicant: THE REGENTS OF THE UNIVERSITY OF MICHIGAN, Ann Arbor, MI (US)

(72) Inventors: Muneesh Tewari, Ann Arbor, MI (US); Sung Won Choi, Ann Arbor, MI (US); Christopher Flora, Temperance, MI (US); Yu Zuo, Ann Arbor, MI (US); Jason Scott Knight, Ann Arbor, MI (US); David G. Frame, Ann Arbor, MI (US)

(21) Appl. No.: 18/706,941

(22) PCT Filed: Nov. 4, 2022
(86) PCT No.: PCT/US2022/049001

§ 371 Date: May 2, 2024

Related U.S. Application Data
(60) Provisional application No. 63/275,482, filed on Nov. 4, 2021.

(51) Int. Cl. G01N 33/68; G01N 33/569; G16B 25/10

(52) U.S. Cl. CPC G01N 33/6863; G01N 33/56972; G01N 33/6893; G16B 25/10; G01N 2800/24

(57) ABSTRACT

Described herein are methods of preventing or treating cytokine release syndrome and/or immune effector cell associated neurotoxicity syndrome (ICANS).

### CROSS-REFERENCE TO RELATED APPLICATIONS

The present application claims the benefit of priority to U.S. Provisional Application No. 63/275,482 filed Nov. 4, 2021, the disclosure of which is incorporated by reference in its entirety.

### STATEMENT ON U.S. GOVERNMENT INTEREST

This invention was made with government support under T32 HL007622 awarded by the National Institutes of Health. The government has certain rights in the invention.

### BACKGROUND

Immunotherapy, including the use of cellular immune therapies or monoclonal antibodies, has become an important tool in the treatment of cancer and neoplasms. T cells expressing chimeric antigens (CAR-T cells) are becoming an option for cellular therapy of cancer. However, while this therapy show promise, treatment with the CAR-T cells may lead to a large, rapid release of cytokines into the blood and cause cytokine release syndrome (CRS) or CAR-T associated neurotoxicity (also referred to herein as ICANS (immune effector cell therapy associated neurotoxicity syndromes)).

CRS is characterized by high fever, hypotension, hypoxia, and/or multi-organ toxicity, and can lead to death. Rarely, CRS can evolve into fulminant hemophagocytic lymphohistiocytosis (HLH), which is characterized by severe immune activation, lymphohistiocytic tissue infiltration and immune-mediated multi-organ failure associated with severe pancytopenia. Neurotoxicity is characterized by damage to nervous tissue that can lead to tremors, encephalopathy, dizziness, or seizures. Both CRS and ICANS are a major cause of death in CAR-T therapy patients, and it is difficult to both predict and to manage these syndromes. Thus, there remains a need in the art to predict which subjects would likely develop CRS and/or ICANs after CAR-T cell therapy.

### SUMMARY

In one aspect, described herein is a method of treating a subject comprising: (a) determining that the subject has (i) an elevated level of calprotectin or citrullinated histone H3 (CitH3) in a plasma sample or (ii) an elevated neutrophil count; and (b) administering an immunotherapy to the subject. In some embodiments, the method further comprises administering an anti-cytokine release syndrome (anti-CRS) therapy to the subject.

In another aspect, described herein is a method of treating a subject comprising: (a) determining that the subject has an elevated level of a protein biomarker of neurotoxicity or of neurotoxicity risk in a plasma sample prior to receiving immunotherapy, wherein the protein biomarker is calprotectin, Secreted Frizzled Related Protein 1 (SFRP1), hepatocyte growth factor (HGF), secreted modular calcium-binding protein 1 (SMOC1), 6-pyruvoyl tetrahydrobiopterin synthase (PTS), low-density lipoprotein (LDL) receptor, tissue plasminogen activator (tPA), tumor necrosis factor-like weak inducer of apoptosis (TWEAK), C-C Motif Chemokine Ligand 18 (CCL18), von Willebrand Factor (vWF), Thrombospondin-related anonymous protein (TRAP), stem cell factor (SCF), Insulin Like Growth Factor Binding Protein 3 (IGFBP3) or Defensin Beta 4A (DEFB4A); and (b) administering a CAR-T cell therapy to the subject. In some embodiments, the method further comprises administering an anti-neurotoxicity agent to the subject.

In another aspect, described herein is a method of treating a subject comprising: (a) administering an immunotherapy to the subject; and (b) determining that the subject has an elevated level, or elevated rate of increase in level, of T-cell immunoglobulin and mucin domain containing 4 (TIMD4) in a plasma sample after said receiving the CAR-T cell therapy. In some embodiments, the method further comprises administering an anti-neurotoxicity agent to the subject.

In another aspect, described herein is a method of treating a subject comprising: (a) administering an immunotherapy to the subject; (b) determining that the subject has an elevated level of: (i) neutrophil/lymphocyte ratio values in a plasma sample from the subject; and/or (ii) neutrophil percentages in a plasma sample from the subject; and (b) administering an anti-neurotoxicity agent to the subject.

In another aspect, described herein is a method of treating a subject comprising (a) administering an immunotherapy to the subject, (b) determining a decreased level of one or more of lymphocyte count, monocyte count, neutrophil percentage, platelet count, and red blood cell count in a blood sample from the subject, and (c) administering an anti-neurotoxicity agent or an anti-cytokine release syndrome (anti-CRS) therapy to the subject.

In some embodiments, the immunotherapy is CAR T cell therapy. In some embodiments, the immunotherapy is an antibody. In some embodiments, the antibody is an anti-CD3 antibody (OKT3), an anti-CD2 antibody (LO-CD2a), an anti-CD20 antibody (rituximab, tositumomab and I¹³¹-tositumomab), an anti-CD28 antibody (TGN1412), an anti-CD52 antibody (alemtuzumab), a CD40 agonist antibody (CP-870,893), a CD3/CD19 bispecific antibody (blinatumomab), an anti-PD-1 antibody (nivolumab), or an anti-IL-2R antibody (basiliximab and daclizumab).

### BRIEF DESCRIPTION OF THE FIGURES

FIG. 1 is a graph providing the results of an Olink proteomic screen to identify plasma proteins differentially expressed in samples of onset cytokine release syndrome (CRS) vs. no CRS.

FIG. 2 provides the results of longitudinal mixed modeling of protein concentrations, which identifies 24 proteins having an increasing trend prior to CRS onset, of which three are neutrophil-related (in bold). Data on the increasing trend in levels of one of the proteins, MPO, in patients developing CRS vs. those not developing CRS, is shown in the graph.

FIG. 3A is a graph showing that baseline (i.e., pre-immunotherapy treatment) blood neutrophil counts are higher in patients who later develop CRS than in patients who do not go on to develop CRS. FIG. 3B is a graph showing that baseline blood neutrophil:lymphocyte ratio is higher in patients who later develop CRS than in patients who do not go on to develop CRS.

FIGS. 4A and 4B are graphs showing that plasma calprotectin (FIG. 4A) and CitH3 (FIG. 4B) are significantly elevated at baseline (i.e., before CAR-T cell infusion) in patients who subsequently go on to develop CRS vs. patients that do not develop CRS.

FIG. 5 is a graph showing the results of an Olink proteomic screen, which identifies plasma SFRP1, HGF, SMOC1, PTS, LDL receptor, tPA, TWEAK, CCL18, vWF, TRAP, SCF, IGFBP3 and DEFB4A as being differentially abundant in specimens at onset of neurotoxicity/ICANS vs. no neurotoxicity.

FIG. 6A provides the results of longitudinal mixed modeling analysis, which identified TIMD4 as a protein that progressively increases in abundance in blood of patients prior to onset of neurotoxicity (shaded circles), but not in patients who do not develop neurotoxicity/ICANS (open, non-shaded circles). NPX on the y-axis represents abundance of TIMD4 protein levels. FIG. 6B is a graph showing that TIMD4 is not elevated in abundance at the time of onset/diagnosis of CRS, and thus is a NT-ICANS-specific biomarker.

FIG. 7 is a graph showing that plasma calprotectin is significantly elevated at baseline (i.e., before CAR-T cell infusion) in patients who subsequently go on to develop neurotoxicity vs. patients who do not develop neurotoxicity.

FIG. 8 is a graph showing that neutrophil/lymphocyte ratio values are significantly higher at baseline (i.e., before CAR-T cell infusion) in patients who subsequently develop Grade 3-4 CRS than those patients who did not develop CRS or developed minimal CRS (Grade 1).

FIG. 9 is a graph showing that lymphocyte percentages were significantly lower at baseline (i.e., before CAR-T cell infusion) in patients who subsequently developed Grade 3-4 CRS than those patients who did not develop CRS or developed minimal CRS (Grade 1).

FIG. 10 is a graph showing that red blood cell distribution was significantly lower at baseline (i.e., before CAR-T cell infusion) in patients who subsequently developed Grade 3-4 CRS than those patients who did not develop CRS.

FIG. 11 is a graph showing that the monocyte/lymphocyte ratios were significantly higher at baseline (i.e., before CAR-T cell infusion) in patients who subsequently developed Grade 3-4 CRS than those patients who did not develop CRS or developed minimal CRS (Grade 1)

FIG. 12 is a graph showing that the mean neutrophil/lymphocyte ratio was significantly higher in the first three days following CAR-T cell infusion in patients who subsequently developed Grade 2 or Grade 3-4 CDR than those patients who did not develop CRS.

FIG. 13 is a graph showing that the mean neutrophil percentage was significantly higher in the first three days following CAR-T cell infusion in patients who subsequently developed Grade 2 or Grade 3-4 CDR than those patients who did not develop CRS.

FIG. 14 is a graph showing that the mean lymphocyte percentage was significantly lower in the first three days following CAR-T cell infusion in patients who subsequently developed Grade 2 or Grade 3-4 CDR than those patients who did not develop CRS.

FIG. 15 is a graph showing that the mean monocyte percentage was significantly lower in the first three days following CAR-T cell infusion in patients who subsequently developed Grade 3-4 CDR than those patients who did not develop CRS.

FIG. 16 is a graph showing that the mean monocyte count was significantly lower in the first three days following CAR-T cell infusion in patients who subsequently developed Grade 3-4 CDR than those patients who did not develop CRS.

FIG. 17 is a graph showing that the mean hemoglobin level was significantly lower in the first three days following CAR-T cell infusion in patients who subsequently developed Grade 3-4 CDR than those patients who did not develop CRS or developed minimal CRS (Grade 1).

FIG. 18 is a graph showing that the mean corpuscular hemoglobin concentration was significantly lower in the first three days following CAR-T cell infusion in patients who subsequently developed Grade 3-4 CDR than those patients who did not develop CRS.

FIG. 19 is a graph showing that the mean lymphocyte percentage was significantly lower in the first three days following CAR-T cell infusion in patients who subsequently developed Grade 2 or Grade 3-4 CDR than those patients who did not develop CRS.

FIG. 20 is a graph showing that the mean hemoglobin level were significantly lower at baseline (i.e., before CAR-T cell infusion) in patients who subsequently developed Grade 3 ICANS than those patients who did not develop ICANS.

FIG. 21 is a graph showing that the mean hematocrit level were significantly lower at baseline (i.e., before CAR-T cell infusion) in patients who subsequently developed Grade 3 ICANS than those patients who did not develop ICANS.

FIG. 22 is a graph showing that red blood cell counts were significantly lower at baseline (i.e., before CAR-T cell infusion) in patients who subsequently developed Grade 3 ICANS than those patients who did not develop ICANS.

FIG. 23 is a graph showing that the neutrophil/lymphocyte ratios were significantly higher in the first three days following CAR-T cell infusion in patients who subsequently developed Grade 1-2 or Grade 3 ICANS than those patients who did not develop ICANS.

FIG. 24 is a graph showing that the neutrophil percentages were significantly higher in the first three days following CAR-T cell infusion in patients who subsequently developed Grade 3 ICANS than those patients who did not develop ICANS or those that developed milder ICANS (Grade 1-2). Patients who developed milder ICANS (Grade 1-2) also had significantly higher neutrophil percentages than those that did not develop ICANS.

FIG. 25 is a graph showing that lymphocyte percentages were significantly lower in the first three days following CAR-T cell infusion in patients who subsequently developed Grade 1-2 or Grade 3 ICANS than those patients who did not develop ICANS.

FIG. 26 is a graph showing that monocyte percentages were significantly lower in the first three days following CAR-T cell infusion in patients who subsequently developed Grade 1-2 or Grade 3 ICANS than those patients who did not develop ICANS.

FIG. 27 is a graph showing that lymphocyte counts were significantly lower in the first three days following CAR-T cell infusion in patients who subsequently developed Grade 1-2 or Grade 3 ICANS than those patients who did not develop ICANS.

FIG. 28 is a graph showing that monocyte counts were significantly lower in the first three days following CAR-T cell infusion in patients who subsequently developed Grade 1-2 or Grade 3 ICANS than those patients who did not develop ICANS.

FIG. 29 is a graph showing that hemoglobin levels were significantly lower in the first three days following CAR-T cell infusion in patients who subsequently developed Grade 3 ICANS than those patients who did not develop ICANS.

FIG. 30 is a graph showing that hematocrit levels were significantly lower in the first three days following CAR-T cell infusion in patients who subsequently developed Grade 3 ICANS than those patients who did not develop ICANS.

FIG. 31 is a graph showing that red blood cell counts were significantly lower in the first three days following CAR-T cell infusion in patients who subsequently developed Grade 3 ICANS than those patients who did not develop ICANS or those that developed milder ICANS (Grade 1-2).

FIG. 32 is a graph showing that platelet counts were significantly lower in the first three days following CAR-T cell infusion in patients who subsequently developed Grade 3 ICANS than those patients who did not develop ICANS.

FIG. 33 is a graph showing that the fold change in platelet count at baseline (i.e., before CAR-T cell infusion) to the first three days following CAR-T cell infusion was significantly decreased from baseline in those patients who subsequently developed Grade 1-2 or Grade 3 ICANS than those patients who did not develop ICANS.

### DETAILED DESCRIPTION

The present disclosure is based on discovery of protein biomarkers useful for the prediction of cytokine release syndrome (CRS) and/or immune effector cell-associated neurotoxicity syndrome (ICANS) in a subject about to undergo CAR-T cell therapy.

The present disclosure is also based on the discovery that continuous temperature monitoring using a wearable sensor in subjects undergoing CAR-T cell therapy enables detection of CRS hours earlier than standard-of-care (SOC) monitoring. It is contemplated that applying mathematical modeling to analyze continuous temperature data in subjects having elevated levels of the protein biomarkers described herein (or elevated neutrophil count) may add additional hours of lead time for anticipating CRS (or ICANS) before it occurs.

### Cytokine Release Syndrome

Cytokine release syndrome, also referred to as cytokine storm, is a form of systemic inflammatory response syndrome that arises as a complication of some diseases or infections, and is also an adverse effect of monoclonal antibody drug or bispecific antibody drug administration, and adoptive T-cell therapies such as CAR-T.

Without wishing to being bound by any particular theory, CRS is triggered by the activation of T cells on engagement of their TCRs or CARs with cognate antigens expressed by tumor cells. The activated T-cells release cytokines and chemokines (including IL-2, soluble IL-2Rα, IFNγ, IL-6, soluble IL-6R, and GM-CSF), as do bystander immune cells, such as monocytes and/or macrophages (which secrete IL-1Ra, IL-10, IL-6, IL-8, CXCL10 (IP-10), CXCL9 (MIG), IFNα, CCL3 (MIP-1α), CCL4 (MIP-1β), and soluble IL-6R), dendritic cells, and others.

CRS can affect any organ system in the body, including cardiovascular, respiratory, integumentary, gastrointestinal, hepatic, renal, hematological, and nervous systems. Patients at high risk of severe CRS include, but are not limited to, those with bulky disease, co-morbidities, and those who develop early onset CRS within three days of cell infusion. High serum levels of cytokines such as IL-6, soluble gp130, IFNγ, IL-15, IL-8, SIL2Rα, IL8, IP10, MCP1, MIG, GM-CSF, TNFα, MIP1β and/or IL-10 after CAR-T cell infusion are associated with subsequent development of severe CRS. In general, there is a balance between the proinflammatory and anti-inflammatory mechanisms, which determines the intensity of the inflammatory response and maintains the immune homeostasis. The proinflammatory and anti-inflammatory cytokines are regulated by complex regulatory networks involving lymphocytes (B cells, T cells, and/or natural killer cells), myeloid cells (macrophages, dendritic cells, and monocytes) and endothelial cells. Moreover, each cytokine also can exert inductive and inhibitive effects on other cytokines, making a cytokine matrix that is responsible for balance regulation.

CRS symptoms can include, without limitation, fever, rapid or disordered heartbeat and breathing, rash, nausea, headache, vomiting, and seizures. CRS can be graded by assessing symptoms and their severities, such as, for example: Grade 1 CRS: Fever, constitutional symptoms; Grade 2 CRS: Hypotension—responds to fluids or one low dose pressor, Hypoxia—responds to <40% 0₂, Organ toxicity; grade 2; Grade 3 CRS: Hypotension—requires multiple pressors or high dose pressors, Hypoxia—requires >40% 0₂, Organ toxicity—grade 3, grade 4 transaminitis; Grade 4 CRS: Mechanical ventilation, Organ toxicity—grade 4, excluding transaminitis. (Lee, et al., Blood 2014; 124:188-195, which is incorporated in its entirety herein by reference.).

CRS and related disorders are not restricted to CAR-T cell therapy, and are also associated with therapeutic monoclonal antibodies such as anti-CD3 (OKT3), anti-CD2 (LO-CD2a), anti-CD20 (rituximab, tositumomab and |131-tositumomab), anti-CD28 (TGN1412), anti-CD52 (alemtuzumab), CD40 agonist antibody (CP-870,893), CD3/CD19 bispecific antibody (blinatumomab), anti-PD-1 (nivolumab), and anti-IL-2R (basiliximab and daclizumab). Immunotherapy agents such as these may also lead to neurotoxicity and/or neurological events in patients.

The onset of CRS toxicity usually occurs within the first week after immunotherapy (e.g., CAR-T cell therapy or antibody) administration, and typically peaks within 1 to 2 weeks of administration. CRS tends to occur earlier in patients treated with certain types of CAR-Ts.

### Immunotherapy Associated Neurotoxicity/ICANS

Immunotherapy (e.g., CAR-T or antibody therapy) associated neurotoxicity/ICANS typically manifests as a toxic encephalopathy, with the earliest signs being diminished attention, language disturbance, and impaired handwriting; other symptoms and signs include confusion, disorientation, agitation, aphasia, somnolence, and tremors. In severe cases of CRES (grade >2), seizures, motor weakness, incontinence, mental obtundation, increased intracranial pressure, papilledema, and cerebral edema can also occur. The manifestation of ICANS can be biphasic; the first phase occurs concurrently with high fever and other CRS symptoms, typically within the first 5 days after cellular immunotherapy, and the second phase occurs after the fever and other CRS symptoms subside, often beyond 5 days after cell infusion.

Similar to CRS, the management of neurotoxicity/ICANS is based on the toxicity grade as outlined in Lee et al. (2018) Biol. Blood Marrow Transplant. December 25. pii: S1083-8791 (18) 31691-4, the contents of which are incorporated herein by reference in their entirety. Grade 1 neurotoxicity/ICANS is primarily managed with supportive care. The head of the patient's bed should be elevated by at least 30 degrees to minimize aspiration risks and to improve cerebral venous flow. A thorough neurological evaluation, including EEG and funduscopic examination to rule out papilledema, of all patients with CRES, regardless of grade, should be performed. Neuroimaging and CSF opening pressure, if available, are much better surrogates of increased intracranial pressure and possible cerebral edema than papilledema; however, lumbar puncture might also be infeasible when patients are restless or have coagulopathy. In patients with an ommaya reservoir, opening pressure can be measured in the supine position with the base of the manometer placed at heart level. Combinations of these techniques should be considered to diagnose increased intracranial pressure and cerebral edema. In particular, repeated neuroimaging, is recommended to detect early signs of cerebral edema in patients with grade 3 or 4 CRES, and in patients with rapid changes in the CRES grade (increase in grade by two levels, for example, grade 1 CRES worsening to grade 3). The clinical status of the patient often dictates the choice of neuroimaging modality: MRI of the brain is preferred, but cannot be performed for unstable or agitated patients, whereas CT can be. The development of cerebral edema in patients treated with CAR-T cells is associated with other acute and clinically significant neurological changes, such as a low CARTOX-10 score and/or seizures. Anti-IL-6 therapy is recommended for patients with grade ≥1 CRES with concurrent CRS; if not associated with CRS, corticosteroids are the preferred treatment for grade ≥2 CRES, and can be tapered after improvement of CRES to grade 1. The optimal duration of corticosteroid therapy remains unknown, although in our experience, short courses of steroids have been associated with resolution of neurological toxicities without impaired antitumor responses. Patients should be monitored closely for recurrence of neurotoxicity symptoms during corticosteroid tapering. Monitoring is required for all patients with grade 4 CRES because they might need mechanical ventilation for airway protection. Non-convulsive and convulsive status epilepticus in these patients should be managed with benzodiazepines and additional antiepileptics (preferably with levetiracetam), as needed. After levetiracetam, phenobarbital is the preferred second antiepileptic for the management of CRES-related seizures: phenytoin and lacosamide are associated with higher risks of cardiovascular adverse effects, therefore, their use in patients with concurrent CRS should be excluded to avoid arrhythmias and hypotension. Grade 3 CRES with raised intracranial pressure should be managed promptly with corticosteroids and acetazolamide; patients who develop grade 4 CRES with cerebral edema should receive high-dose corticosteroids, hyperventilation, and hyperosmolar therapy.

### Treatment Methods

In one aspect, disclosed herein is a method of treating a subject comprising determining that the subject has an elevated level of (i) calprotectin or citrullinated histone H3 (CitH3) in a sample; or (ii) an elevated neutrophil count; administering an immunotherapy to the subject; and administering an immunotherapy to the subject. In some embodiments, the method further comprises administering a cytokine release syndrome (CRS) therapy to the subject. In some embodiments, an elevated level of calprotectin or CitH3, or an elevated neutrophil count identifies the subject as likely to develop cytokine release syndrome (CRS). In this regard, subjects identified as having elevated levels of calprotectin and/or CitH3 and/or an elevated neutrophil count relative to a reference standard would be candidates for early CRS therapy (i.e., anti-CRS therapy administered shortly after (or concurrently with) the immunotherapy. In some embodiments, the anti-CRS therapy is administered prior to the immunotherapy as a preventive measure.

In another aspect, disclosed herein is a method of treating a subject comprising determining that the subject has an elevated level of a protein marker of neurotoxicity (or neurotoxicity risk) in a sample prior to receiving an immunotherapy, wherein the protein biomarker is calprotectin, is calprotectin, Secreted Frizzled Related Protein 1 (SFRP1), hepatocyte growth factor (HGF), secreted modular calcium-binding protein 1 (SMOC1), 6-pyruvoyl tetrahydrobiopterin synthase (PTS), low-density lipoprotein (LDL) receptor, tissue plasminogen activator (tPA), tumor necrosis factor-like weak inducer of apoptosis (TWEAK), C-C Motif Chemokine Ligand 18 (CCL18), von Willebrand Factor (vWF), Thrombospondin-related anonymous protein (TRAP), stem cell factor (SCF), Insulin Like Growth Factor Binding Protein 3 (IGFBP3) or Defensin Beta 4A (DEFB4A); and administering an immunotherapy to the subject. In some embodiments, the method further comprises administering an anti-neurotoxicity therapy to the subject. In some embodiments, an elevated level of the protein biomarker identifies the subject as likely to develop neurotoxicity. In this regard, subjects identified as having elevated levels of the protein biomarker relative to a reference standard would be candidates for early anti-neurotoxicity therapy (i.e., anti-neurotoxicity therapy administered shortly after (or concurrently with) the immunotherapy). In some embodiments, the anti-neurotoxicity therapy is administered prior to the immunotherapy as a preventive measure.

In another aspect, disclosed herein is a method of treating a subject comprising administering an immunotherapy to the subject; determining that the subject has an elevated level, or elevated rate of increase, of T-cell immunoglobulin and mucin domain containing 4 (TIMD4) in a sample after receiving said immunotherapy; and administering an anti-neurotoxicity agent to the subject. In some embodiments, an elevated level of TIMD4 identifies the subject as likely to develop neurotoxicity. In this regard, subjects identified as having elevated levels of TIMD4 relative to a reference standard would be candidates for early anti-neurotoxicity therapy (i.e., anti-neurotoxicity therapy administered shortly after (or concurrently with) the immunotherapy).

In another aspect, disclosed herein is a method of treating a subject comprising administering an immunotherapy to the subject; determining that the subject has an elevated level, or elevated rate of increase, of one or more of CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, and LTBP3 in a sample after receiving said immunotherapy; and administering an anti-neurotoxicity agent to the subject. In some embodiments, an elevated level of one or more of CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, and LTBP3 identifies the subject as likely to develop neurotoxicity. In this regard, subjects identified as having elevated levels of one or more of CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, and LTBP3 relative to a reference standard would be candidates for early anti-neurotoxicity therapy (i.e., anti-neurotoxicity therapy administered shortly after (or concurrently with) the immunotherapy).

In another aspect, disclosed herein is a method of treating a subject comprising administering an immunotherapy to the subject; determining that the subject has a decreased level, or elevated rate of decrease, of one or more of TANK, ITGA11, CNTNAP2, ITGB2FUT8, CLEC4A, PROC, CR2, TIE1, MMP9, PAM, CASP3, SLAMF1, CD244, Gal3, ISLR2, EIF4B, BACH1, CDH17, IGFBP3, CD6, PIK3AP1, uPA1, AXIN1, QPCT, AKT1S1, TDGF1, DNER, DAPP1, COMP, RBKS, PGLYRP1, CRADD, AARSD1, and SPRY2 in a sample after receiving said immunotherapy; and administering an anti-neurotoxicity agent to the subject. In some embodiments, a decreased level of one or more of TANK, ITGA11, CNTNAP2, ITGB2FUT8, CLEC4A, PROC, CR2, TIE1, MMP9, PAM, CASP3, SLAMF1, CD244, Gal3, ISLR2, EIF4B, BACH1, CDH17, IGFBP3, CD6, PIK3AP1, uPA1, AXIN1, QPCT, AKT1S1, TDGF1, DNER, DAPP1, COMP, RBKS, PGLYRP1, CRADD, AARSD1, and SPRY2 identifies the subject as likely to develop neurotoxicity. In this regard, subjects identified as having decreased levels of one or more of TANK, ITGA11, CNTNAP2, ITGB2FUT8, CLEC4A, PROC, CR2, TIE1, MMP9, PAM, CASP3, SLAMF1, CD244, Gal3, ISLR2, EIF4B, BACH1, CDH17, IGFBP3, CD6, PIK3AP1, uPA1, AXIN1, QPCT, AKT1S1, TDGF1, DNER, DAPP1, COMP, RBKS, PGLYRP1, CRADD, AARSD1, and SPRY2 relative to a reference standard would be candidates for early anti-neurotoxicity therapy (i.e., anti-neurotoxicity therapy administered shortly after (or concurrently with) the immunotherapy).

In some embodiments, disclosed herein is a method of treating a subject comprising: determining that the subject has an elevated level of monocyte/lymphocyte ratio in a blood sample; and administering an immunotherapy to the subject.

In another aspect, described herein is a method of treating a subject comprising administering an immunotherapy to the subject; determining that the subject has an elevated level of: (i) neutrophil/lymphocyte ratio values in a blood sample from the subject; and/or (ii) neutrophil percentages in a blood sample from the subject; and administering an anti-neurotoxicity agent to the subject.

In another aspect, disclosed herein is a method of treatment comprising administering an immunotherapy to the subject, determining decreased levels of one or more of lymphocyte count, monocyte count, neutrophil percentage, platelet count, and red blood cell count from a blood sample from the subject after receiving the immunotherapy, and administering an anti-neurotoxicity agent or an anti-CDR therapy to the subject. In some embodiments, the method comprises identifying decreased levels of lymphocyte count, monocyte count, red blood cell count, neutrophil percentage and platelet count in a blood sample from the subject after receiving the immunotherapy. In some embodiments, the comprises identifying decreased levels of lymphocyte count, monocyte count, RBC count and platelet count in a blood sample from the subject after receiving the immunotherapy. In some embodiments, the method comprises determining the level of one or more of lymphocyte count, monocyte count, RBC count, neutrophil percentage and platelet count in a blood sample from a subject before and after administering the immunotherapy to the subject.

In some embodiments, a decreased level of one or more of lymphocyte count, monocyte count, red blood cell count, neutrophil percentage and platelet count is an amount that is ≥10% decreased compared to baseline. In some embodiments, the decreased level of one or more of lymphocyte count, monocyte count, red blood cell count, neutrophil percentage and platelet count in the sample comprises an amount that is at least a 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or 100% decreased compared to baseline. In some embodiments, the decreased level of one or more of lymphocyte count, monocyte count, red blood cell count, neutrophil percentage and platelet count in the sample comprises an amount that is above a threshold that is set in the range of 1-4 standard deviations change from baseline in a reference control group of patients that did not experience neurotoxicity, and/or in a reference control group of control patients that experienced only mild neurotoxicity. In some embodiments, the decreased level of one or more of lymphocyte count, monocyte count, red blood cell count, neutrophil percentage and platelet count in the sample comprises an amount that is a decrease from baseline that is reflected in the slope of a linear fit line of lymphocyte count, monocyte count, red blood cell count, neutrophil percentage and/or platelet count values over time being above a threshold set in the range of +0.1 to +1. In some embodiments, the decreased level of lymphocyte count, monocyte count, red blood cell count, neutrophil percentage and/or platelet count in the sample comprises an amount that is a decrease from baseline that is reflected as the slope of a linear fit line of lymphocyte count, monocyte count, red blood cell count, neutrophil percentage and/or platelet count values over time that is above a threshold that is set in the range of 1-4 standard deviations above the mean slope in a reference control group of patients that did not experience neurotoxicity, and/or in a reference control group of control patients that experienced only mild neurotoxicity.

In some embodiments, a decreased level of one or more of TANK, ITGA11, CNTNAP2, ITGB2FUT8, CLEC4A, PROC, CR2, TIE1, MMP9, PAM, CASP3, SLAMF1, CD244, Gal3, ISLR2, EIF4B, BACH1, CDH17, IGFBP3, CD6, PIK3AP1, uPA1, AXIN1, QPCT, AKT1S1, TDGF1, DNER, DAPP1, COMP, RBKS, PGLYRP1, CRADD, AARSD1, and SPRY2 is an amount that is ≥10% decreased compared to baseline. In some embodiments, the decreased level of one or more of TANK, ITGA11, CNTNAP2, ITGB2FUT8, CLEC4A, PROC, CR2, TIE1, MMP9, PAM, CASP3, SLAMF1, CD244, Gal3, ISLR2, EIF4B, BACH1, CDH17, IGFBP3, CD6, PIK3AP1, uPA1, AXIN1, QPCT, AKT1S1, TDGF1, DNER, DAPP1, COMP, RBKS, PGLYRP1, CRADD, AARSD1, and SPRY2 in the sample comprises an amount that is at least a 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or 100% decreased compared to baseline. In some embodiments, the decreased level of one or more of TANK, ITGA11, CNTNAP2, ITGB2FUT8, CLEC4A, PROC, CR2, TIE1, MMP9, PAM, CASP3, SLAMF1, CD244, Gal3, ISLR2, EIF4B, BACH1, CDH17, IGFBP3, CD6, PIK3AP1, uPA1, AXIN1, QPCT, AKT1S1, TDGF1, DNER, DAPP1, COMP, RBKS, PGLYRP1, CRADD, AARSD1, and SPRY2 in the sample comprises an amount that is above a threshold that is set in the range of 1-4 standard deviations change from baseline in a reference control group of patients that did not experience neurotoxicity, and/or in a reference control group of control patients that experienced only mild neurotoxicity. In some embodiments, the decreased level of one or more of TANK, ITGA11, CNTNAP2, ITGB2FUT8, CLEC4A, PROC, CR2, TIE1, MMP9, PAM, CASP3, SLAMF1, CD244, Gal3, ISLR2, EIF4B, BACH1, CDH17, IGFBP3, CD6, PIK3AP1, uPA1, AXIN1, QPCT, AKT1S1, TDGF1, DNER, DAPP1, COMP, RBKS, PGLYRP1, CRADD, AARSD1, and SPRY2i n the sample comprises an amount that is a decrease from baseline that is reflected in the slope of a linear fit line of TANK, ITGA11, CNTNAP2, ITGB2FUT8, CLEC4A, PROC, CR2, TIE1, MMP9, PAM, CASP3, SLAMF1, CD244, Gal3, ISLR2, EIF4B, BACH1, CDH17, IGFBP3, CD6, PIK3AP1, uPA1, AXIN1, QPCT, AKT1S1, TDGF1, DNER, DAPP1, COMP, RBKS, PGLYRP1, CRADD, AARSD1, and/or SPRY2 values over time being above a threshold set in the range of +0.1 to +1. In some embodiments, the decreased level of TANK, ITGA11, CNTNAP2, ITGB2FUT8, CLEC4A, PROC, CR2, TIE1, MMP9, PAM, CASP3, SLAMF1, CD244, Gal3, ISLR2, EIF4B, BACH1, CDH17, IGFBP3, CD6, PIK3AP1, uPA1, AXIN1, QPCT, AKT1S1, TDGF1, DNER, DAPP1, COMP, RBKS, PGLYRP1, CRADD, AARSD1, and/or SPRY2 in the sample comprises an amount that is a decrease from baseline that is reflected as the slope of a linear fit line of TANK, ITGA11, CNTNAP2, ITGB2FUT8, CLEC4A, PROC, CR2, TIE1, MMP9, PAM, CASP3, SLAMF1, CD244, Gal3, ISLR2, EIF4B, BACH1, CDH17, IGFBP3, CD6, PIK3AP1, uPA1, AXIN1, QPCT, AKT1S1, TDGF1, DNER, DAPP1, COMP, RBKS, PGLYRP1, CRADD, AARSD1, and/or SPRY2 values over time that is above a threshold that is set in the range of 1-4 standard deviations above the mean slope in a reference control group of patients that did not experience neurotoxicity, and/or in a reference control group of control patients that experienced only mild neurotoxicity.

In some embodiments, the immunotherapy is CAR T cell therapy. In some embodiments, the immunotherapy is an antibody. In some embodiments, the antibody is an anti-CD3 antibody (OKT3), an anti-CD2 antibody (LO-CD2a), an anti-CD20 antibody (rituximab, tositumomab and |131-tositumomab), an anti-CD28 antibody (TGN1412), an anti-CD52 antibody (alemtuzumab), a CD40 agonist antibody (CP-870,893), a CD3/CD19 bispecific antibody (blinatumomab), an anti-PD-1 antibody (nivolumab), or an anti-IL-2R antibody (basiliximab and daclizumab).

As used herein, the term “treatment” refers to an approach for obtaining a beneficial or a desired result including, but not limited to, a therapeutic benefit or prevention of a condition, e.g., a side effect (such as an unwanted effect as described herein). The terms “treatment”, “treating”, and “ameliorating” are used interchangeably herein. In some embodiments, a therapeutic benefit is obtained by eradication or amelioration of the underlying disorder being treated. In some embodiments, a therapeutic benefit is obtained by reduction of, eradication, or amelioration of one or more of the symptoms, e.g., physiological symptoms, associated with the underlying disorder such that an improvement, e.g., change, is observed in the subject. In some embodiments, the subject can still be afflicted with the underlying disorder. In some embodiments, treatment comprises prevention of a condition, e.g., a side effect (such as an unwanted side effect from a therapy). Treatment or prevention of a condition or a side effect need not be a complete treatment or prevention of the condition or side effect.

In any of the methods described herein, in some embodiments, the sample is a blood sample. In some embodiments, the sample is a plasma sample. In some embodiments, the sample is a serum sample.

The determining step of the methods described herein optionally comprises comparing the measurement of calprotectin, CitH3, SFRP1, HGF, SMOC1, PTS, LDL receptor, tPA, TWEAK, CCL18, VWF, TRAP, SCF, IGFBP3, DEFB4A (or TIMD4) to a reference measurement of calprotectin, CitH3, SFRP1, HGF, SMOC1, PTS, LDL receptor, tPA, TWEAK, CCL18, VWF, TRAP, SCF, IGFBP3, DEFB4A (or TIMD4), and scoring the measurement from the sample as elevated based on statistical analysis or a ratio relative to the reference measurement. In some embodiments, the reference measurement comprises at least one of the following (a) calprotectin, CitH3, SFRP1, HGF, SMOC1, PTS, LDL receptor, tPA, TWEAK, CCL18, VWF, TRAP, SCF, IGFBP3, DEFB4A (or TIMD4) protein levels in a blood sample from a subject that is not suffering from cancer, (b) calprotectin, CitH3, SFRP1, HGF, SMOC1, PTS, LDL receptor, tPA, TWEAK, CCL18, VWF, TRAP, SCF, IGFBP3, DEFB4A (or TIMD4) protein levels in a collection of comparable blood samples from cancer patients treated with a similar therapy (e.g. CAR-T cell therapy, other immune therapies that can trigger CRS or neurotoxicity syndromes) but who did not develop CRS and/or developed only mild CRS (for CRS biomarkers), or who did not develop neurotoxicity and/or developed only mild neurotoxicity (for neurotoxicity biomarkers); or (c) calprotectin, CitH3, SFRP1, HGF, SMOC1, PTS, LDL receptor, tPA, TWEAK, CCL18, VWF, TRAP, SCF, IGFBP3, DEFB4A (or TIMD4) protein level in an arbitrary standard optionally further including statistical distribution information for the multiple measurements, such as standard deviation. In some embodiments, the methods described herein comprise comparing the level of a protein biomarker described herein (i.e., calprotectin, CitH3, SFRP1, HGF, SMOC1, PTS, LDL receptor, tPA, TWEAK, CCL18, VWF, TRAP, SCF, IGFBP3, DEFB4A and/or TIMD4) in a blood sample from the subject to the level of the protein biomarker in a blood sample from a healthy subject, or from a reference control set of comparable patients who developed no and/or mild CRS and/or neurotoxicity, wherein an elevated level compared to the reference standard identifies the subject as a subject that would benefit from treatment with an anti-CRS therapy (and/or anti-neurotoxicity therapy and/or anti-neutrophil therapy).

In some embodiments, the methods described herein comprise comparing the level of calprotectin and/or CitH3 in a blood sample from the subject to the level of expression in a healthy subject, or from a reference control set of comparable patients who developed no and/or mild CRS and/or neurotoxicity, wherein an elevated level compared to the sample from the healthy subject and/or to the reference control set of patients identifies the subject as likely to benefit from early treatment with an anti-CRS therapy.

In some embodiments, the methods described herein comprise comparing the level of calprotectin, SFRP1, HGF, SMOC1, PTS, LDL receptor, tPA, TWEAK, CCL18, vWF, TRAP, SCF, IGFBP3, and/or DEFB4A in a blood sample from the subject to the level of SFRP1, HGF, SMOC1, PTS, LDL receptor, tPA, TWEAK, CCL18, vWF, TRAP, SCF, IGFBP3, and/or DEFB4A in a healthy subject, or from a reference control set of comparable patients who developed no and/or mild CRS and/or neurotoxicity, wherein an elevated level compared to the sample from the healthy subject and/or to the reference control set of patients identifies the subject as a subject that would benefit from treatment with an anti-neurotoxicity therapy (and/or anti-neutrophil therapy).

In some embodiments, the elevated level of calprotectin in the sample comprises an amount that is above a threshold that is set in the range of 1-4 standard deviations above the mean value in a reference control group of patients that did not experience CRS, and/or in a reference control group that only developed mild (Grade 1) CRS (for the instance in which calprotectin is used as a CRS biomarker); or in a reference control group that did not experience neurotoxicity, and/or in a reference control group that only developed mild neurotoxicity (for the instance in which calprotectin is used as a neurotoxicity biomarker). In some embodiments, the elevated level of calprotectin in the sample comprises an amount that is ≥1,000 ng/mL. In some embodiments, the elevated level of calprotectin in the sample comprises an amount that is at least 1,000 ng/mL, or at least 1,500 ng/mL, or at least 2,000 ng/mL, or at least 2,500 ng/mL, or at least 3,000 ng/mL, or at least 3,500 ng/mL, or at least 4,000 ng/mL, or at least 4,500 ng/mL, or at least 5,000 ng/mL, or at least 5,500 ng/mL, or at least 6,000 ng/mL, or at least 6,500 ng/mL, or at least 7,000 ng/mL, or at least 7,500 ng/mL, or at least 8,000 ng/mL, or at least 8,500 ng/mL, or at least 9,000 ng/mL, or at least 9,500 ng/mL, or at least 10,000 ng/mL.

In some embodiments, the elevated level of HGF in the sample comprises an amount that is above a threshold that is set in the range of 1-4 standard deviations above the mean value in a reference control group of patients that did not experience CRS, and/or in a reference control group that only developed mild (Grade 1) CRS (for the instance in which HGF is used as a CRS biomarker); or in a reference control group that did not experience neurotoxicity, and/or in a reference control group that only developed mild neurotoxicity (for the instance in which HGF is used as a neurotoxicity biomarker).

In some embodiments, the elevated level of SMOC1 in the sample comprises an amount that is above a threshold that is set in the range of 1-4 standard deviations above the mean value in a reference control group of patients that did not experience CRS, and/or in a reference control group that only developed mild (Grade 1) CRS (for the instance in which SMOC1 is used as a CRS biomarker); or in a reference control group that did not experience neurotoxicity, and/or in a reference control group that only developed mild neurotoxicity (for the instance in which SMOC1 is used as a neurotoxicity biomarker).

In some embodiments, the elevated level of PTS in the sample comprises an amount that is above a threshold that is set in the range of 1-4 standard deviations above the mean value in a reference control group of patients that did not experience CRS, and/or in a reference control group that only developed mild (Grade 1) CRS (for the instance in which PTS is used as a CRS biomarker); or in a reference control group that did not experience neurotoxicity, and/or in a reference control group that only developed mild neurotoxicity (for the instance in which PTS is used as a neurotoxicity biomarker).

In some embodiments, the elevated level of LDL receptor in the sample comprises an amount that is above a threshold that is set in the range of 1-4 standard deviations above the mean value in a reference control group of patients that did not experience CRS, and/or in a reference control group that only developed mild (Grade 1) CRS (for the instance in which LDL receptor is used as a CRS biomarker); or in a reference control group that did not experience neurotoxicity, and/or in a reference control group that only developed mild neurotoxicity (for the instance in which LDL receptor is used as a neurotoxicity biomarker).

In some embodiments, the elevated level of tPA in the sample comprises an amount that is above a threshold that is set in the range of 1-4 standard deviations above the mean value in a reference control group of patients that did not experience CRS, and/or in a reference control group that only developed mild (Grade 1) CRS (for the instance in which tPA is used as a CRS biomarker); or in a reference control group that did not experience neurotoxicity, and/or in a reference control group that only developed mild neurotoxicity (for the instance in which tPA is used as a neurotoxicity biomarker).

In some embodiments, the elevated level of TWEAK in the sample comprises an amount that is above a threshold that is set in the range of 1-4 standard deviations above the mean value in a reference control group of patients that did not experience CRS, and/or in a reference control group that only developed mild (Grade 1) CRS (for the instance in which TWEAK is used as a CRS biomarker); or in a reference control group that did not experience neurotoxicity, and/or in a reference control group that only developed mild neurotoxicity (for the instance in which TWEAK is used as a neurotoxicity biomarker).

In some embodiments, the elevated level of CCL18 in the sample comprises an amount that is above a threshold that is set in the range of 1-4 standard deviations above the mean value in a reference control group of patients that did not experience CRS, and/or in a reference control group that only developed mild (Grade 1) CRS (for the instance in which CCL18 is used as a CRS biomarker); or in a reference control group that did not experience neurotoxicity, and/or in a reference control group that only developed mild neurotoxicity (for the instance in which CCL18 is used as a neurotoxicity biomarker).

In some embodiments, the elevated level of vWF in the sample comprises an amount that is above a threshold that is set in the range of 1-4 standard deviations above the mean value in a reference control group of patients that did not experience CRS, and/or in a reference control group that only developed mild (Grade 1) CRS (for the instance in which vWF is used as a CRS biomarker); or in a reference control group that did not experience neurotoxicity, and/or in a reference control group that only developed mild neurotoxicity (for the instance in which vWF is used as a neurotoxicity biomarker).

In some embodiments, the elevated level of TRAP in the sample comprises an amount that is above a threshold that is set in the range of 1-4 standard deviations above the mean value in a reference control group of patients that did not experience CRS, and/or in a reference control group that only developed mild (Grade 1) CRS (for the instance in which TRAP is used as a CRS biomarker); or in a reference control group that did not experience neurotoxicity, and/or in a reference control group that only developed mild neurotoxicity (for the instance in which TRAP is used as a neurotoxicity biomarker).

In some embodiments, the elevated level of SCF in the sample comprises an amount that is above a threshold that is set in the range of 1-4 standard deviations above the mean value in a reference control group of patients that did not experience CRS, and/or in a reference control group that only developed mild (Grade 1) CRS (for the instance in which SCF is used as a CRS biomarker); or in a reference control group that did not experience neurotoxicity, and/or in a reference control group that only developed mild neurotoxicity (for the instance in which SCF is used as a neurotoxicity biomarker).

In some embodiments, the elevated level of IFGBP3 in the sample comprises an amount that is above a threshold that is set in the range of 1-4 standard deviations above the mean value in a reference control group of patients that did not experience CRS, and/or in a reference control group that only developed mild (Grade 1) CRS (for the instance in which UGFBP3 is used as a CRS biomarker); or in a reference control group that did not experience neurotoxicity, and/or in a reference control group that only developed mild neurotoxicity (for the instance in which IGFBP3 is used as a neurotoxicity biomarker).

In some embodiments, the elevated level of DEFB4A in the sample comprises an amount that is above a threshold that is set in the range of 1-4 standard deviations above the mean value in a reference control group of patients that did not experience CRS, and/or in a reference control group that only developed mild (Grade 1) CRS (for the instance in which DEFB4A is used as a CRS biomarker); or in a reference control group that did not experience neurotoxicity, and/or in a reference control group that only developed mild neurotoxicity (for the instance in which DEFB4A is used as a neurotoxicity biomarker)

In some embodiments, the elevated level of CitH3 in the sample comprises an amount that is above a threshold that is set in the range of 1-4 standard deviations above the mean value in a reference control group of patients that did not experience CRS, and/or in a reference control group that only developed mild (Grade 1) CRS. In some embodiments, the elevated level of CitH3 in the sample comprises an amount that is ≥5 ng/ml. In some embodiments, the elevated level of CitH3 in the sample comprises an amount that is at least 5 ng/ml, or at least 6 ng/ml, or at least 7 ng/ml, or at least 8 ng/ml, or at least 9 ng/ml, or at least 10 ng/ml, or at least 11 ng/ml, or at least 12 ng/ml, or at least 13 ng/ml, or at least 14 ng/ml, or at least 15 ng/ml, or at least 16 ng/ml, or at least 17 ng/ml, or at least 18 ng/ml, or at least 19 ng/ml, or at least 20 ng/ml.

In some embodiments, the elevated level of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) in the sample comprises an amount that is ≥10% increase compared to baseline. In some embodiments, the elevated level of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) in the sample comprises an amount that is at least a 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or 100% increase compared to baseline. In some embodiments, the elevated level of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) in the sample comprises an amount that is above a threshold that is set in the range of 1-4 standard deviations change from baseline in a reference control group of patients that did not experience neurotoxicity, and/or in a reference control group of control patients that experienced only mild neurotoxicity. In some embodiments, the elevated level of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) in the sample comprises an amount that is an increase from baseline that is reflected in the slope of a linear fit line of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) values over time being above a threshold set in the range of +0.1 to +1. In some embodiments, the elevated level of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) in the sample comprises an amount that is an increase from baseline that is reflected as the slope of a linear fit line of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) values over time that is above a threshold that is set in the range of 1-4 standard deviations above the mean slope in a reference control group of patients that did not experience neurotoxicity, and/or in a reference control group of control patients that experienced only mild neurotoxicity.

In some embodiments, the methods described herein comprise comparing the neutrophil count in a sample from the subject before (i.e., at baseline) and after immunotherapy, and scoring the measurement from the sample as elevated based on statistical analysis or a ratio relative to the reference measurement. In some embodiments, the methods described herein comprise comparing the neutrophil count in a sample from the subject before and after immunotherapy, wherein an elevated neutrophil count in a sample from the subject after immunotherapy compared to the neutrophil count in a sample from the subject before immunotherapy identifies the subject as likely to benefit from early treatment with an anti-CRS therapy.

In some embodiments, the subject has a baseline neutrophil count ≥1,000 cells/μl. In some embodiments, subject has a baseline neutrophil count that is at least 1,000 cells/μl, or at least 1,500 cells/μl, or at least 2,000 cells/μl, or at least 2,500 cells/μl, or at least 3,000 cells/μl, or at least 3,500 cells/μl, or at least 4,000 cells/μl, or at least 4,500 cells/μl, or at least 5,000 cells/μl, or at least 5,500 cells/μl, or at least 6,000 cells/μl, or at least 6,500 cells/μl, or at least 7,000 cells/μl, or at least 7,500 cells/μl, or at least 8,000 cells/μl, or at least 8,500 cells/μl, or at least 9,000 cells/μl, or at least 9,500 cells/μl, or at least 10,000 cells/μl. In some embodiments, the subject has a baseline neutrophil count is above a threshold set in the range of 1-4 standard deviations above the mean value of baseline neutrophil count in a control group of patients that did not experience CRS, and/or in a reference control group of control patients that experienced only mild CRS (Grade 1).

In some embodiments, the subject has a baseline blood neutrophil count:lymphocyte count ratio that is above a threshold set in the range of 1-4 standard deviations above the mean value of this ratio in a reference control group of patients that did not experience CRS, and/or in a reference control group of control patients that experienced only mild CRS (Grade 1). In some embodiments, the subject has a baseline blood neutrophil count:lymphocyte count ratio that is above a threshold set in the range of 20%-500% above the mean value of this ratio in a reference control group of patients that did not experience CRS, and/or in a reference control group of control patients that experienced only mild CRS (Grade 1). In some embodiments, the subject has a baseline blood neutrophil count:lymphocyte count ratio that is ≥2. In some embodiments, the subject has a baseline blood neutrophil count:lymphocyte count ratio that is above 2, or above 3, or above 4, or above 5, or above 10, or above 20, or above 30, or above 40, or above 50.

The methods described herein may optionally comprise the step of identifying a subject as not being a candidate for treatment with an anti-CRS therapy or an anti-neurotoxicity therapy, if the level of calprotectin, CitH3, SFRP1, HGF, SMOC1, PTS, LDL receptor, tPA, TWEAK, CCL18, VWF, TRAP, SCF, IGFBP3, DEFB4A (or TIMD4) (or neutrophil count) in the blood sample from the subject is lower than the reference standard.

In some embodiments, the determining step is performed between 1-14 days prior to administering the immunotherapy to the subject. In some embodiments, the determining step is performed 1, 2, 3, 4, 5, 6, 7, 8, 9, 10, 11, 12, 13, or 14 days prior to administering the immunotherapy to the subject. In some embodiments, the determining step is performed 1-7 days prior to administering the immunotherapy to the subject. In some embodiments, the determining step is performed 1, 2, 3, 4, 5, 6, or 7 days prior to administering the immunotherapy to the subject. In some embodiments, the determining step is performed 7 days prior to administering the immunotherapy to the subject.

In some embodiments, the methods described herein comprise comparing the change in level from baseline, or rate of change from baseline in blood sample(s) from the subject to the change in level from baseline or rate of change from baseline, in a reference control group of patients that did not experience neurotoxicity, and/or in a reference control group of control patients that experienced only mild neurotoxicity, wherein an elevated level compared to the samples from the reference control group identifies the subject as a subject that would benefit from treatment with an anti-neurotoxicity therapy. In some embodiments, the baseline level of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) is determined between 1-14 days prior to administering the immunotherapy to the subject. In some embodiments, the baseline level of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) is determined 1, 2, 3, 4, 5, 6, 7, 8, 9, 10, 11, 12, 13, or 14 days prior to administering the immunotherapy to the subject. In some embodiments, the baseline level of TIMD4 is determined 1-7 days prior to administering the immunotherapy to the subject. In some embodiments, the baseline level of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) is determined 1, 2, 3, 4, 5, 6, or 7 days prior to administering the immunotherapy to the subject. In some embodiments, the baseline level of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) is determined 7 days prior to administering the immunotherapy to the subject.

In some embodiments, the rate of change of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) from baseline is determined by comparing a baseline level of TIMD4 in a plasma subject from the subject obtained 1, 2, 3, 4, 5, 6, 7, 8, 9, 10, 11, 12, 13, or 14 days prior to administration of the immunotherapy to a level of TIMD4 (or CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, or LTBP3) in a plasma sample from the subject obtained 1, 2, 3, 4, 5, 6, 7, 8, 9, 10, 11, 12, 13, or 14 days after administration of the immunotherapy.

In some embodiments, the determining step is performed between within 24 hours after administering the immunotherapy to the subject. In some embodiments, the determining step is performed 30 minutes or 1, 2, 3, 4, 5, 6, 7, 8, 9, 10, 11, 12, 13, 14, 15, 16, 17, 18, 19, 20, 21, 22, 23 or 24 hours after administering the immunotherapy to the subject.

In some embodiments, the methods described herein further comprise monitoring the subject for symptoms indicative of the onset of CRS/ICANS before administering an anti-CRS therapy or an anti-neurotoxicity therapy to the subject. In some embodiments, the monitoring step comprises assessing the body temperature of the subject over time, assessing vital signs at least every 4 hours, and daily review of organ systems, physical exam, complete blood count with differential, complete metabolic profile, coagulation profiles, and/or measurement of serum CRP and ferritin levels.

In some embodiments, the methods described herein comprise continuous temperature monitoring of the subject and administering the anti-CRS therapy (or the anti-neurotoxicity therapy) to the subject when the subject has a body temperature 37° C. or higher (e.g., 37° C., 38° C., 39° C., 40° C., 41° C. or 42° C. or higher) following the administration of the CAR-T cell therapy.

### Anti-CRS and Anti-Neurotoxicity Therapies

Standard of care therapies for CRS include treatment with an IL-6 inhibitor, an IL-6 receptor (IL-6R) inhibitor (e.g., tocilizumab or siltuximab), bazedoxifene, a sgp130 blocker, vasoactive medications, corticosteroids, immunosuppressive agents, and mechanical ventilation. Additional therapies for CRS are disclosed, for example, in International Application Publication No. WO 2014/011984, which is hereby incorporated by reference.

Tocilizumab is a humanized, immunoglobulin G1kappa anti-human IL-6R monoclonal antibody. See, e.g., id. Tocilizumab blocks binding of IL-6 to soluble and membrane bound IL-6 receptors (IL-6Rs) and thus inhibits classical and trans-IL-6 signaling. In some embodiments, tocilizumab is administered at a dose of about 4-12 mg/kg, e.g., about 4-8 mg/kg for adults and about 8-12 mg/kg for pediatric subjects, e.g., administered over the course of 1 hour.

In some embodiments, the anti-CRS therapy comprises administering an inhibitor of IL-6 signaling, e.g., an inhibitor of IL-6 or IL-6 receptor, to the subject. In some embodiments, the inhibitor is an anti-IL-6 antibody, e.g., siltuximab. In some embodiments, the inhibitor comprises a soluble gp130 (sgp130) or a fragment thereof that is capable of blocking IL-6 signaling. In some embodiments, the sgp130 or fragment thereof is fused to a heterologous domain, e.g., an Fc domain, e.g., is a gp130-Fc fusion protein such as FE301. In embodiments, the inhibitor of IL-6 signaling comprises an antibody, e.g., an antibody to the IL-6 receptor, such as sarilumab, olokizumab (CDP6038), elsilimomab, sirukumab (CNTO 136), ALD518/BMS-945429, ARGX-109, or FM101. In some embodiments, the inhibitor of IL-6 signaling comprises a small molecule such as CPSI-2364.

In some embodiments, the anti-neurotoxicity therapy comprises administering dexamethasone, methylprednisone, a corticosteroid, defibrotide and or an anti-cytokine agent to the subject. In some embodiments, the anti-cytokine agent is an anti-GM-CSF agent, or an anti-GM-CSF receptor agent, an anti-IL-1 receptor agent, an anti-IL-1 agent, an anti-IL6 receptor agent an anti-IL-6 agent.

In some embodiments, the methods further comprise administering a steroid to the subject.

In some embodiments, the anti-CRS therapy (or anti-neurotoxicity therapy) is administered within 5 hours (e.g., within 30 minutes, or within 1, 2, 3, 4, or 5 hours after administering the immunotherapy to the subject. In some embodiments, the anti-CRS therapy (or anti-neurotoxicity therapy) is administered to the subject within 1-3 hours (e.g., within 30 minutes or within 1, 2, or 3 hours) of administration of the immunotherapy being administered to the subject. In some the anti-CRS therapy (or anti-neurotoxicity therapy) is administered concurrently with (at the same time as) the immunotherapy.

In some embodiments, the anti-CRS therapy (or anti-neurotoxicity therapy) is administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), a dose of the immunotherapy.

In some embodiments, the anti-CRS therapy (or anti-neurotoxicity therapy) is administered up to 5 days (e.g., 1, 2, 3, 4 or 5 days) before administering the immunotherapy to the subject.

In some embodiments, the methods described herein comprise administering an anti-neutrophil therapy/agent to the subject. Exemplary anti-neutrophil agents include, but are not limited to, colchicine, Selectin antagonists, Anti-integrin antibodies, Inhibitors of CXCR1, CXCR2, leukotriene B4 receptor 1 (BLT1) or C5a receptor (C5aR), NETosis inhibitors, Anti-IL-17A monoclonal antibodies, Ustekinumab, an anti-p40 antibody blocking the common subunit of the IL-23 and IL-12 receptors, Extracorporeal granulocytapheresis, JAK inhibitors, Anti-GM-CSF, Anti-GM-CSF Receptor, Metoprolol and Calprotectin inhibitors.

### Cancer

In any of the methods described herein, the subject is suffering from cancer. The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

In some embodiments, the cancer is a hematologic cancer. Examples of hematological (or hematogenous) cancers include, but are not limited to, leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

In some embodiments, the cancer is a solid tumor. Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include, but are not limited to, fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

A subject “responds” to treatment if a parameter of a cancer (e.g., cancer cell growth, proliferation and/or survival) in the subject is slowed or reduced by a detectable amount, e.g., about 5%, 10%, 20%, 30%, 40%, 50%, 60%, 70%, 80%, 90% or more as determined by any appropriate measure, e.g., by mass, cell count or volume. In one example, a subject responds to treatment if the subject experiences a life expectancy extended by about 5%, 10%, 20%, 30%, 40%, 50% or more beyond the life expectancy predicted if no treatment is administered. In another example, a subject responds to treatment, if the subject has an increased disease-free survival, overall survival or increased time to progression. Several methods can be used to determine if a patient responds to a treatment including, for example, criteria provided by NCCN Clinical Practice Guidelines in Oncology (NCCN Guidelines®). For example, in the context of B-ALL, a complete response or complete responder, may involve one or more of: <5% BM blast, >1000 neutrophil/ANC (/μL). >100,000 platelets (/μL) with no circulating blasts or extramedullary disease (no lymphadenopathy, splenomegaly, skin/gum infiltration/testicular mass/CNS involvement), Trilineage hematopoiesis, and no recurrence for 4 weeks. A partial responder may involve one or more of >50% reduction in BM blast, >1000 neutrophil/ANC (/μL). >100,000 platelets (/μL). A non-responder can show disease progression, e.g., >25% in BM blasts.

### CAR-T-Cells

The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain, e.g., comprise a chimeric fusion protein. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule of the CAR is the zeta chain associated with the T cell receptor complex (e.g., CD3 zeta). In one aspect, the cytoplasmic signaling domain comprises a primary signaling domain (e.g., a primary signaling domain of CD3-zeta).

In one aspect, the cytoplasmic signaling domain further comprises one or more functional signaling domains derived from at least one costimulatory molecule as defined below. In one aspect, the costimulatory molecule is chosen from the costimulatory molecules described herein, e.g., 4-1BB (i.e., CD137), CD27, and/or CD28. In one aspect, the CAR comprises a chimeric fusion protein comprising an extracellular antigen binding domain, a transmembrane domain and an intracellular signaling domain comprising a functional signaling domain derived from a stimulatory molecule. In one aspect, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one aspect, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one aspect, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., a scFv) during cellular processing and localization of the CAR-To the cellular membrane.

In some embodiments, the CAR-T cell comprises an antigen binding domain that binds to a tumor antigen selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In some embodiments, the CAR-T cell therapy is administered in a single infusion or a split-dose infusion. In some embodiments, the CAR-T cell therapy is administered in a single infusion. In some embodiments, the CAR-T cell therapy is administered at a dosage of about 1×10⁸, 2×10⁸, 3×10⁸, 4×10⁸, 5×10⁸, 6×10⁸, 7×10⁸, 8×10⁸, 9×10⁸ cells, e.g., about 5×10⁸ cells, e.g., about 5×10⁸ cells in a single infusion. In some embodiments, the CAR-T cell therapy is administered at a dosage of about 0.1×10⁸, 0.2×10⁸, 0.3×10⁸, 0.4×10⁸, 0.5×10⁸, 0.6×10⁸, 0.7×10⁸, 0.8×10⁸, 0.9×10⁸, 1×10⁸, 1.5×10⁸, 2×10⁸, 2.5×10⁸, 3×10⁸, 3.5×10⁸, 4×10⁸, 5×10⁸, 6×10⁸, 7×10⁸, 8×10⁸, 9×10⁸ or 1×10⁹, e.g., 0.6-6×10⁸ or 1-5×10⁸, in a single infusion.

### EXAMPLES

### Materials and Methods

Proteomic screening: Proteomic analysis was done on plasma specimens using Olink proximity extension assay technology, which enables quantification of hundreds of protein targets in a plasma sample. Specifically, longitudinal specimens from a cohort of n=29 patients experiencing a range of CRS or NT (including no CRS or NT) grades were analyzed for abundance of 460 known proteins, spanning biological domains of inflammation, immune response, cardiovascular, cardiometabolic, and neural-related.

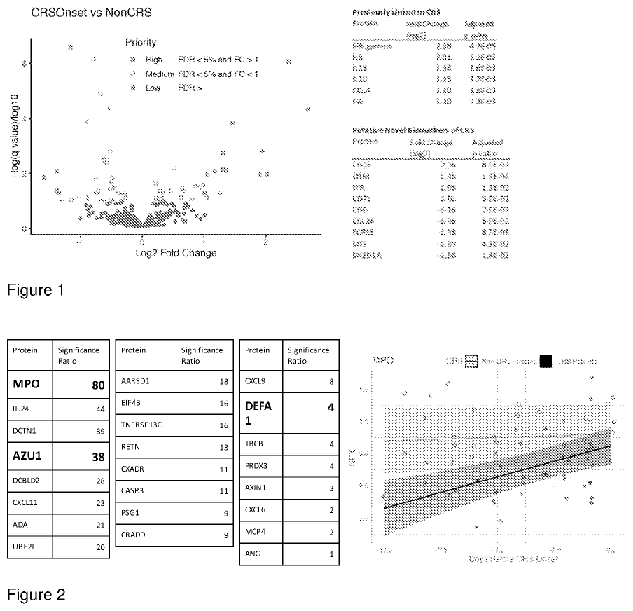

Proteomic data analysis to identify CRS biomarkers: One approach to the data analyses was to identify protein markers in plasma that were elevated at the time of diagnosis of CRS (FIG. 1) or that show a longitudinally changing trend prior to diagnosis of CRS (FIG. 2). In addition to the identified candidate biomarkers, proteins expressed in neutrophils appeared prominently, which included CD33 (expressed in myeloid cells; FIG. 1) and three proteins highly related to neutrophil function (MPO, AZU1, DEFA1; FIG. 2). This led to the hypothesis that activation of the innate immune system through neutrophils may underlie CRS.

### Example 1—Biomarkers Predictive of Cytokine Release Syndrome (CRS)

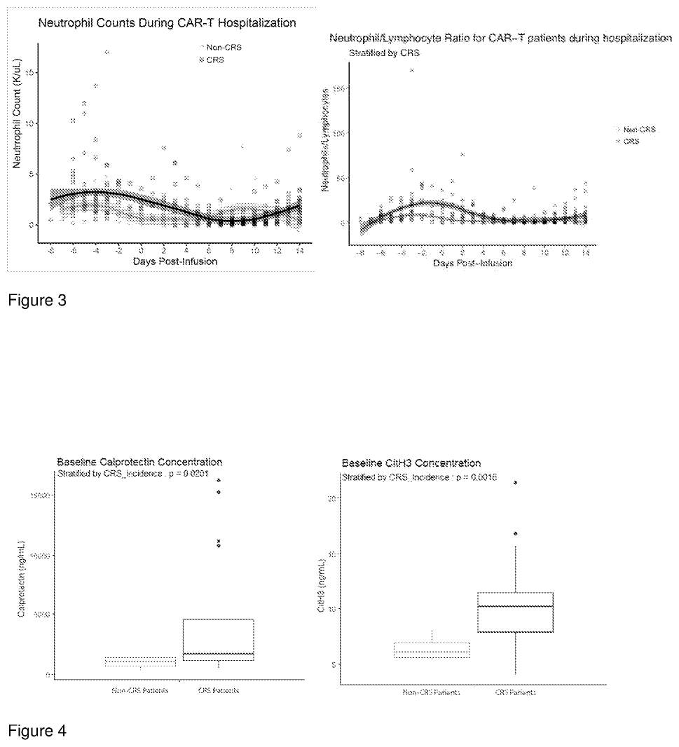

As a next step to investigate the possibility of neutrophil involvement with CRS, longitudinal data on neutrophil counts was retrieved for the patients in the study and found that absolute neutrophil counts (ANC) differed between patients who developed CRS vs. No CRS. Importantly, this difference was present at baseline, before the infusion of CAR-T cells, on “day 0”, as well as on the days preceding day 0 (i.e., day −1, day −2, day −3, day −4, day −5, day −6 and day −7) (FIG. 3A). The blood neutrophil:lymphocyte ratio is also higher at baseline in patients who later develop CRS than in patients who do not go on to develop CRS FIG. 3B).

It is known that neutrophils can undergo an activation process called NETosis, which involves extrusion outside the cell of neutrophil DNA along with proteins decorating it, to form neutrophil extracellular traps (NETs). This is part of an innate immune response to bacterial infection, but has also been known to occur in settings not associated with infection, referred to sometime as “sterile NETosis”. NETosis is believed to help trap pathogens locally in the NETs that are formed, but has also been suggested to have other functions that help amplify the overall immune response of an individual.

CitH3, a known biomarker of NETosis, and calprotectin, a neutrophil-associated marker also implicated in NETosis, were measured in our cohort of CAR-T patient plasma specimens. This analysis showed that calprotectin and to an even greater extent, CitH3, were elevated at baseline (i.e., prior to CAR-T cell infusion) in patients who later developed CRS vs. No CRS (FIG. 4).

### Example 2—Biomarkers Predictive of Immune Effector Cell-Associated Neurotoxicity Syndrome (ICANS)

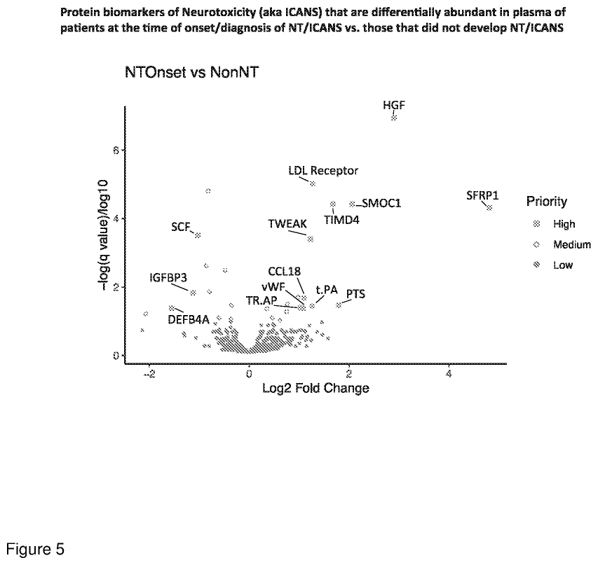

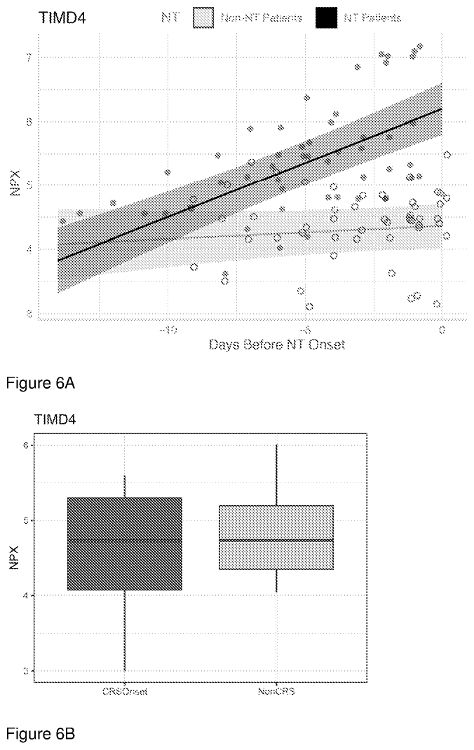

A similar data analyses as described in Example 1 was performed to identify biomarkers associated with CAR-T therapy associated NT (also known as immune effector cell-associated neurotoxicity syndrome (ICANS)). From the proteomic analysis, 14 proteins were identified as being significantly differentially abundant between plasma samples at the onset of NT vs. plasma samples in patients without NT (FIG. 5). Additionally, from a longitudinal mixed modeling analysis that identified proteins showing a progressive change in abundance prior to the onset of NT, one protein that fit this pattern was identified, TIMD4 (FIG. 6A). FIG. 6B demonstrates that TIMD4 is not elevated in abundance at the time of onset/diagnosis of CRS, and thus is a NT-ICANS-specific biomarker.

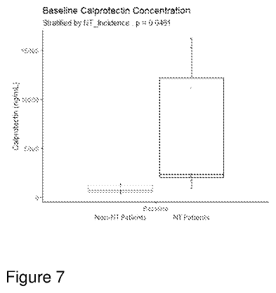

Furthermore, two biomarkers of NETosis and neutrophil activation, CitH3 and calprotectin respectively, were examined as predictors of NT. It was found that calprotectin showed a significantly elevated level in baseline (i.e., pre-CAR-T infusion) plasma specimens in patients who went on to later develop NT vs No NT (FIG. 7). This was not the case for CitH3 (data not shown). These data nominate calprotectin as a baseline biomarker for identification of patients who are highest risk for developing NT from immunotherapy.

### Example 3—Further Analyses of Blood Biomarkers Predictive of CRS

The following analyses was conducted on the same group of patients identified in the Materials and Methods section above.

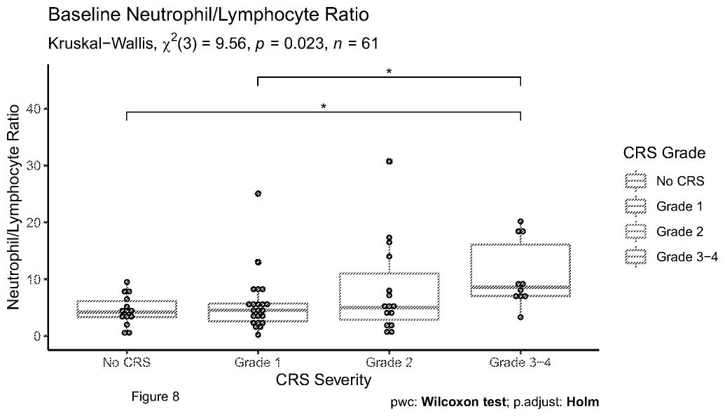

Neutrophil and lymphocyte count data from patient electronic health records of patients receiving CAR-T cell therapies were analyzed, and the mean neutrophil/lymphocyte ratio was calculated at pre-treatment baseline before CAR-T cell infusion. As shown in FIG. 8, patients who went on to develop Grade 3-4 CRS had significantly higher neutrophil/lymphocyte ratio values than those who developed no CRS or minimal CRS (Grade 1). The asterisks represent p<0.05.

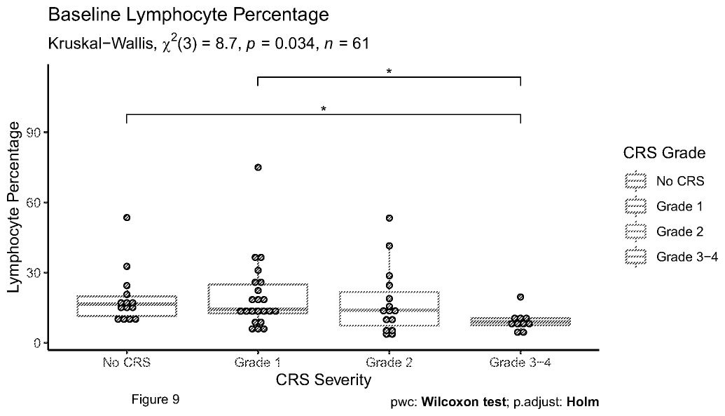

Lymphocyte count data from patient electronic health records of patients receiving CAR-T cell therapies was analyzed, and the mean lymphocyte count at pre-treatment baseline before CAR-T cell infusion was calculated. As shown in FIG. 9, patients who went on to develop Grade 3-4 CRS had significantly lower lymphocyte percentages than those who developed no CRS or minimal CRS (Grade 1). The asterisks represent p<0.05.

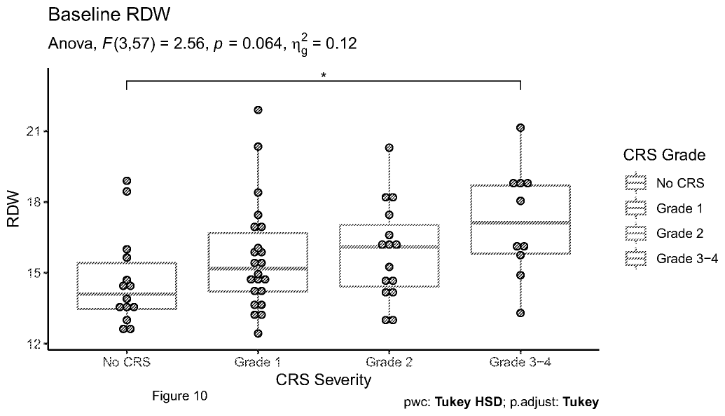

Blood count data from patient electronic health records of patients receiving CAR-T cell therapies was analyzed, and the mean red blood cell distribution width at pre-treatment baseline before CAR-T cell infusion was calculated. As shown in FIG. 10, patients who went on to develop Grade 3-4 CRS had significantly lower red blood cell distribution than those that did not develop CRS. The asterisk represents p<0.05.

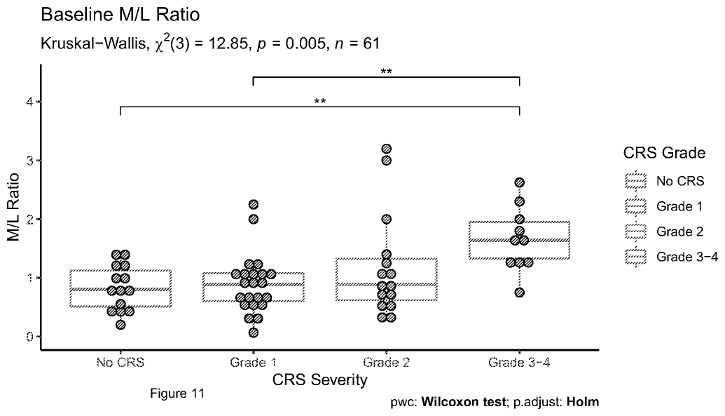

Monocyte and lymphocyte count data was analyzed from patient electronic health records of patients receiving CAR-T cell therapies, and the mean monocyte/lymphocyte ratio at pre-treatment baseline before CAR-T cell infusion was calculated. As shown in FIG. 11, patients who went on to develop Grade 3-4 CRS had significantly higher monocyte/lymphocyte ratios than those who developed no CRS or minimal CRS (Grade 1). The asterisks represent p<0.01.

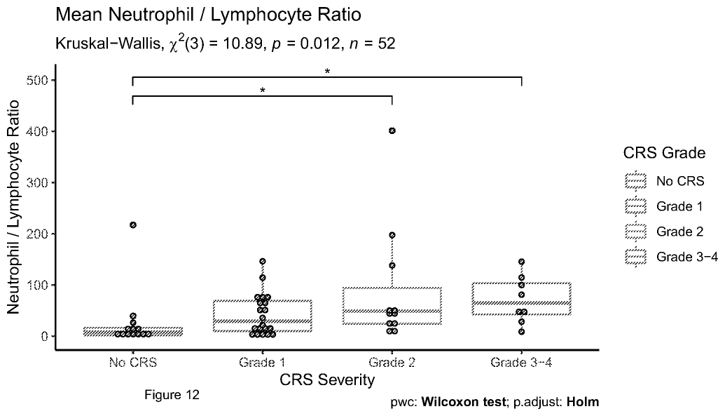

Neutrophil and lymphocyte count data from patient electronic health records of patients receiving CAR-T cell therapies was analyzed, and the mean neutrophil/lymphocyte ratio from the first three days following CAR-T cell infusion was calculated. As shown in FIG. 12, patients who developed Grade 2 or Grade 3-4 CRS had significantly higher neutrophil/lymphocyte ratio values than those that did not develop CRS. The asterisks represent p<0.05.

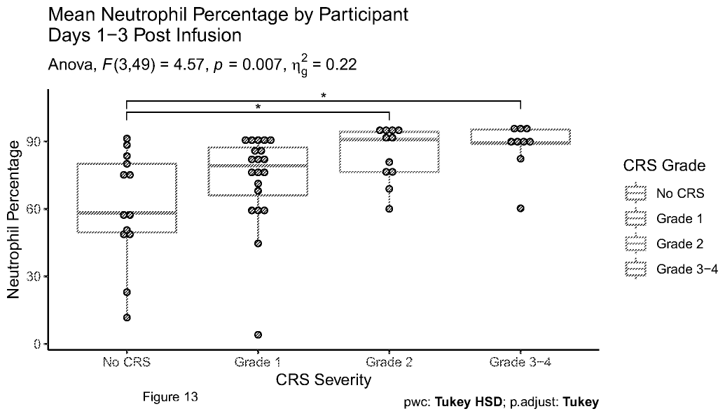

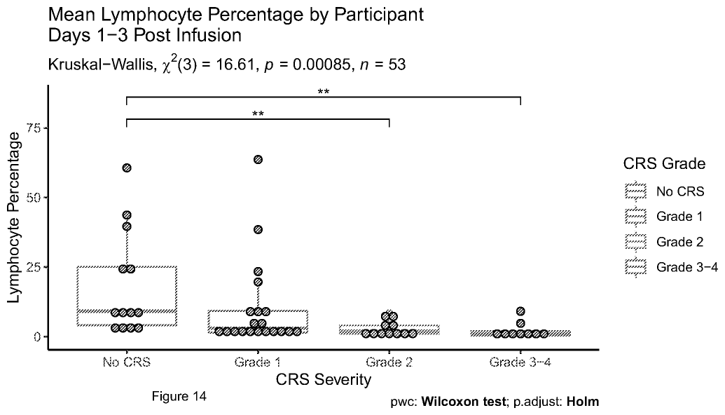

Neutrophil percentage and lymphocyte percentage data from patient electronic health records of patients receiving CAR-T cell therapies was analyzed, and the mean neutrophil percentage from the first three days following CAR-T cell infusion was calculated. As shown in FIGS. 13 and 14, respectively, patients who developed Grade 2 or Grade 3-4 CRS had significantly higher neutrophil percentages and significantly lower lymphocyte percentages than those that did not develop CRS. The asterisk represents p<0.05.

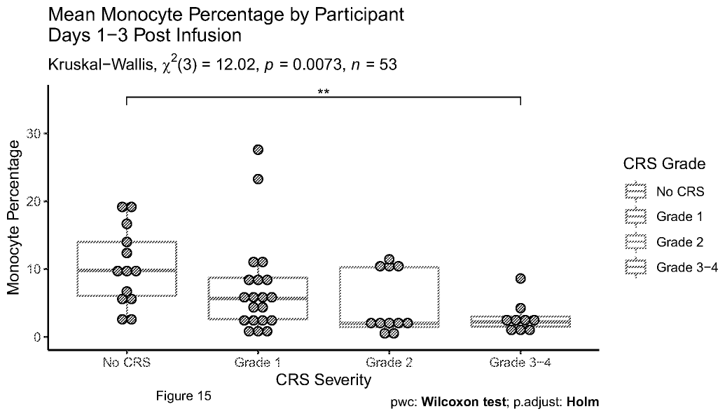

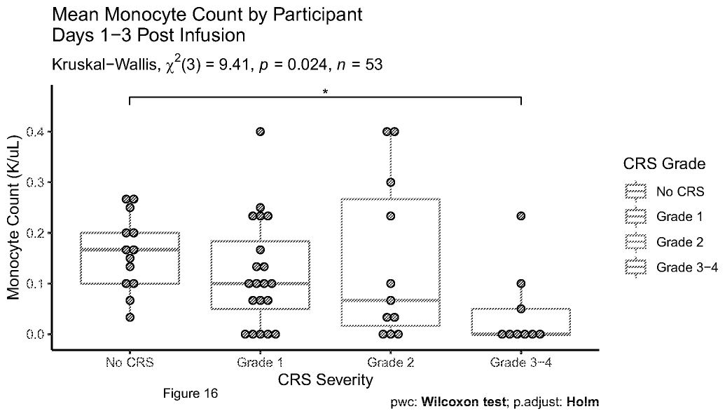

Monocyte percentage and monocyte count data from patient electronic health records of patients receiving CAR-T cell therapies was analyzed, and the mean monocyte percentage and mean monocyte count from the first three days following CAR-T cell infusion was analyzed. As shown in FIGS. 15 and 16, patients who developed Grade 3-4 CRS had significantly lower monocyte percentages and significantly lower monocyte count than those that did not develop CRS. The asterisk represents p<0.01.

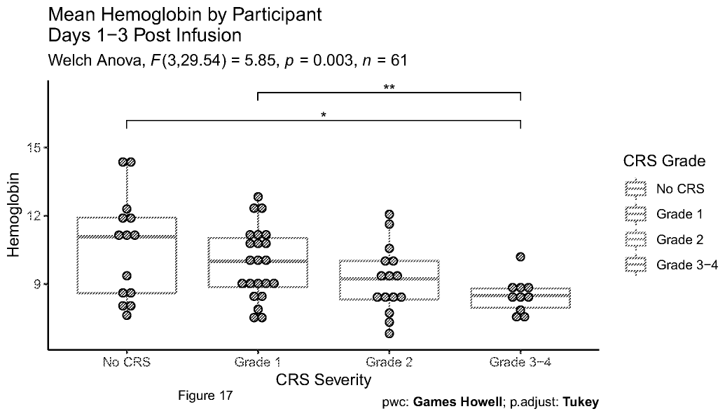

Blood count data from patient electronic health records of patients receiving CAR-T cell therapies was analyzed, and the mean hemoglobin level from the first three days following CAR-T cell infusion was calculated. As shown in FIG. 17, patients who developed Grade 3-4 CRS had significantly lower hemoglobin levels than those who developed no CRS or minimal CRS (Grade 1). The asterisks represent p<0.05 and p<0.01 respectively.

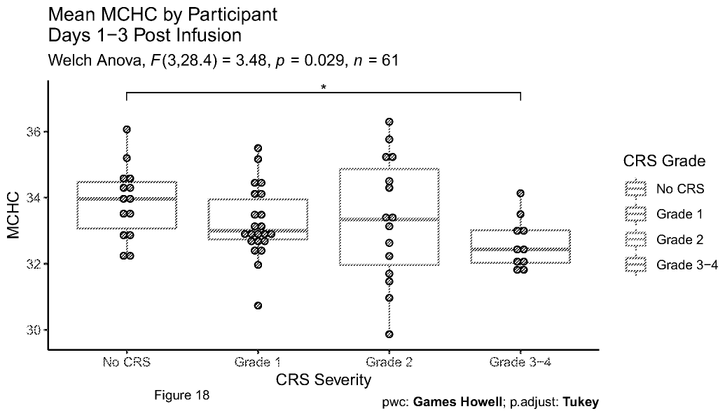

Blood count data from patient electronic health records of patients receiving CAR-T cell therapies was analyzed, and the mean corpuscular hemoglobin concentration from the first three days following CAR-T cell infusion was calculated. As shown in FIG. 18, patients who developed Grade 3-4 CRS had significantly lower hemoglobin levels than that did not develop CRS. The asterisk represents p<0.05.

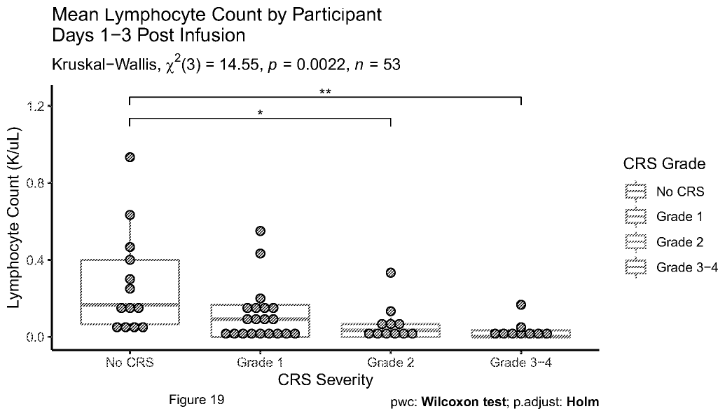

Lymphocyte count data from patient electronic health records of patients receiving CAR-T cell therapies was analyzed, and the mean lymphocyte count from the first three days following CAR-T cell infusion was calculated. As shown in FIG. 19, patients who developed Grade 2 or Grade 3-4 CRS had significantly lower monocyte counts than those that did not develop CRS. The asterisks represent p<0.05 and p<0.01 respectively.

### Example 4—Further Analyses of Blood Biomarkers Predictive of ICANS

The following analyses was conducted on the same group of patients identified in the Materials and Methods section above.

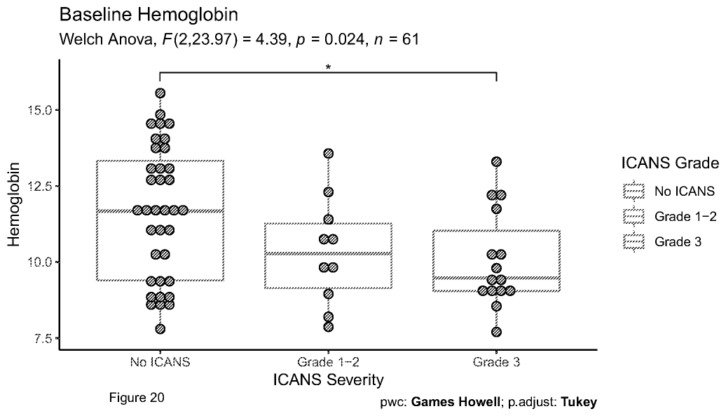

Blood count data from patient electronic health records of patients receiving CAR-T cell therapies was analyzed, and the mean hemoglobin level at pre-treatment baseline before CAR-T cell infusion was calculated. As shown in FIG. 20, patients who went on to develop Grade 3 ICANS had significantly lower hemoglobin levels than those that did not develop ICANS. The asterisk represents p<0.05.

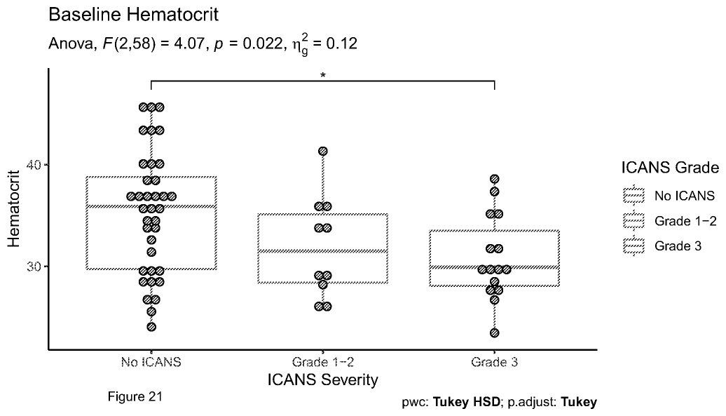

Blood count data from patient electronic health records of patients receiving CAR-T cell therapies was analyzed, and the mean hematocrit level at pre-treatment baseline before CAR-T cell infusion was calculated. As shown in FIG. 21, patients who went on to develop Grade 3 ICANS had significantly lower hematocrit levels than those that did not develop ICANS. The asterisk represents p<0.05.

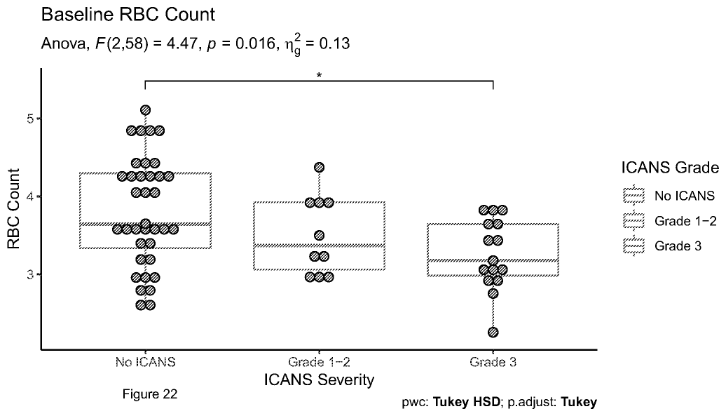

Blood count data from patient electronic health records of patients receiving CAR-T cell therapies was analyzed, and the mean red blood cell count at pre-treatment baseline before CAR-T cell infusion was calculated. As shown in FIG. 22, patients who went on to develop Grade 3 ICANS had significantly lower red blood cell counts than those that did not develop ICANS. The asterisk represents p<0.05.

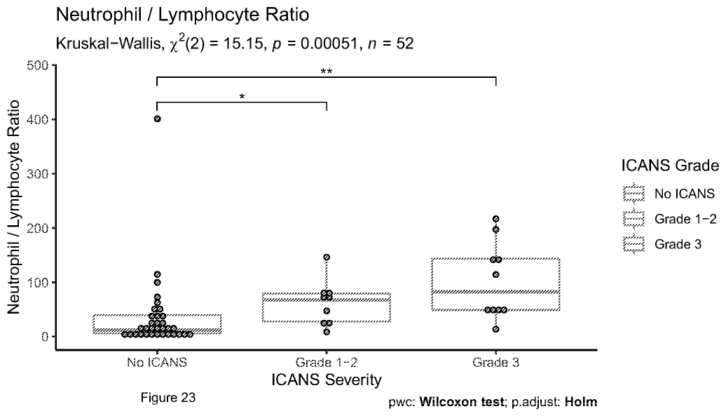

Neutrophil and lymphocyte count data from patient electronic health records of patients receiving CAR-T cell therapies was analyzed, and the mean neutrophil/lymphocyte ratio from the first three days following CAR-T cell infusion was calculated. As shown in FIG. 23, patients that went on to develop Grade 1-2 or Grade 3 ICANS had significantly higher neutrophil/lymphocyte ratio values than those that did not develop ICANS. The asterisks represent p<0.05 and p<0.01, respectively.

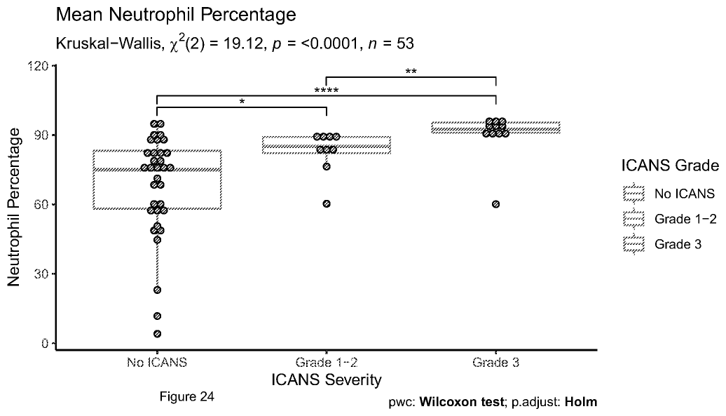

Neutrophil percentage data from patient electronic health records of patients receiving CAR-T cell therapies was analyzed, and the mean neutrophil percentage from the first three days following CAR-T cell infusion was calculated. As shown in FIG. 24, patients that went on to develop Grade 3 ICANS had significantly higher neutrophil percentages than those who developed no ICANS or milder ICANS (Grade 1-2). Patients who developed milder ICANS (Grade 1-2) also had significantly higher neutrophil percentages than those that did not develop ICANS. The asterisks represent p<0.05, p<0.01, and p<0.0001 respectively.

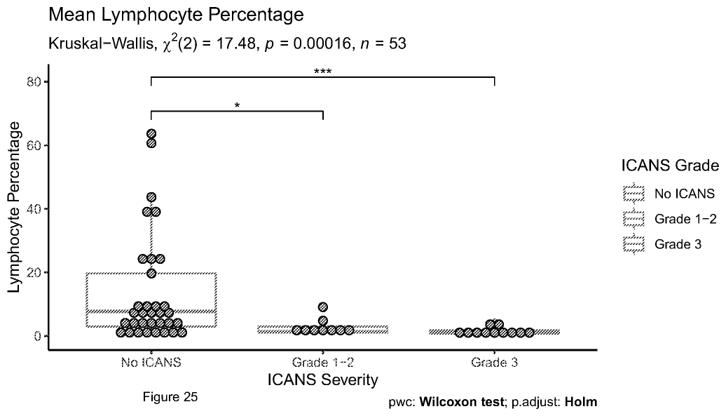

Lymphocyte percentage data from patient electronic health records of patients receiving CAR-T cell therapies was analyzed, and the mean lymphocyte percentage from the first three days following CAR-T cell infusion was calculated. As shown in FIG. 25, patients that went on to develop Grade 1-2 or Grade 3 ICANS had significantly lower lymphocyte percentages than those that did not develop ICANS. The asterisks represent p<0.05 and p<0.001 respectively.

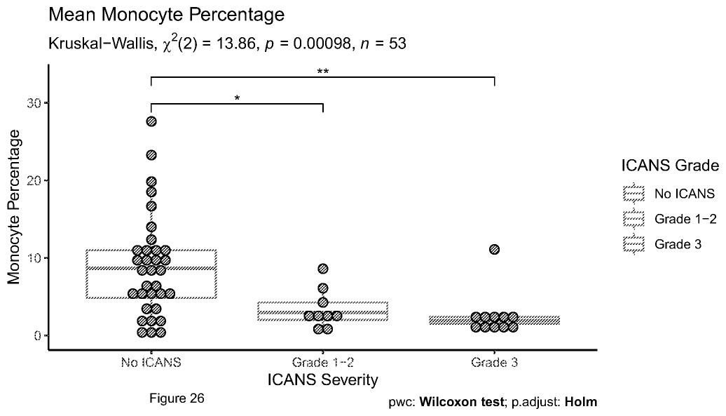

Monocyte percentage data from patient electronic health records of patients receiving CAR-T cell therapies was analyzed, and the mean monocyte percentage from the first three days following CAR-T cell infusion was calculated. As shown in FIG. 26, patients that went on to develop Grade 1-2 or Grade 3 ICANS had significantly lower monocyte percentages than those that did not develop ICANS. The asterisks represent p<0.05 and p<0.01 respectively.

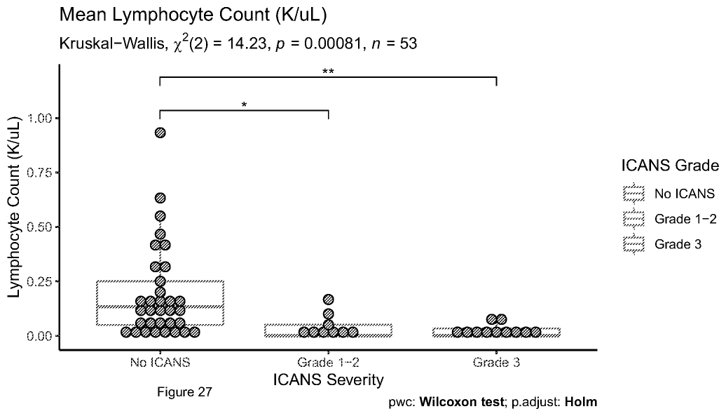

Lymphocyte count data from patient electronic health records of patients receiving CAR-T cell therapies was analyzed, and the mean lymphocyte count from the first three days following CAR-T cell infusion was calculated. As shown in FIG. 27, patients that went on to develop Grade 1-2 or Grade 3 ICANS had significantly lower lymphocyte counts than those that did not develop ICANS. The asterisks represent p<0.05 and p<0.01 respectively.

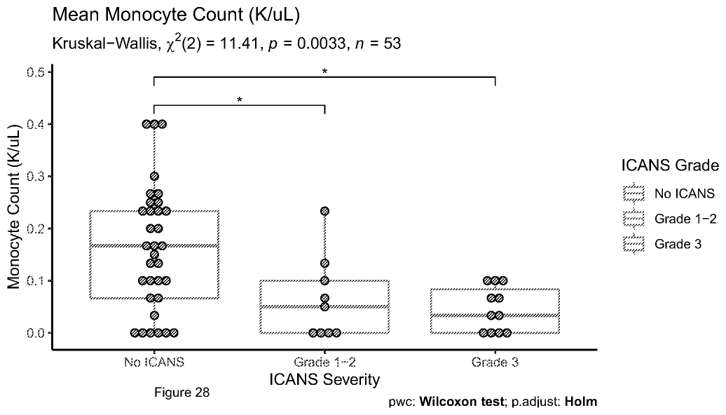

Monocyte count data from patient electronic health records of patients receiving CAR-T cell therapies was analyzed, and the mean monocyte count from the first three days following CAR-T cell infusion was calculated. As shown in FIG. 28, patients that went on to develop Grade 1-2 or Grade 3 ICANS had significantly lower monocyte counts than those that did not develop ICANS. The asterisks represent p<0.05.

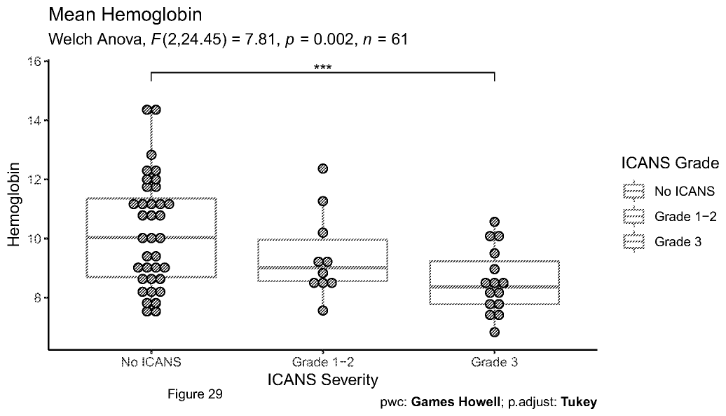

Monocyte count data from patient electronic health records of patients receiving CAR-T cell therapies was analyzed, and the mean monocyte count from the first three days following CAR-T cell infusion was calculated. As shown in FIG. 29, patients that went on to develop Grade 1-2 or Grade 3 ICANS had significantly lower monocyte counts than those that did not develop ICANS. The asterisks represent p<0.05.

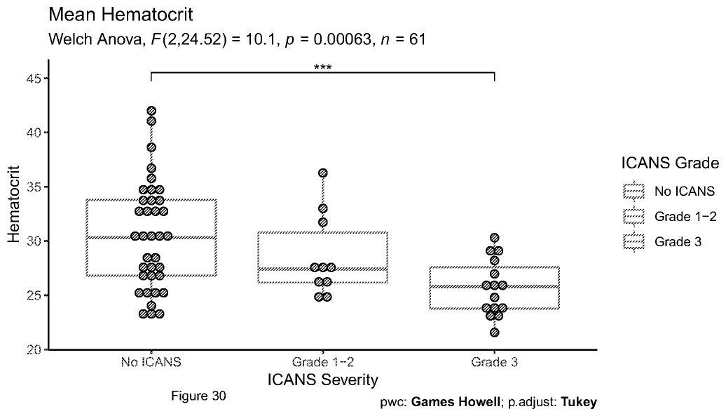

Blood count data from patient electronic health records of patients receiving CAR-T cell therapies was analyzed, and the mean hematocrit level from the first three days following CAR-T cell infusion was calculated. As shown in FIG. 30, patients that went on to develop Grade 3 ICANS had significantly lower hematocrit levels than those that did not develop ICANS. The asterisk represents p<0.001.

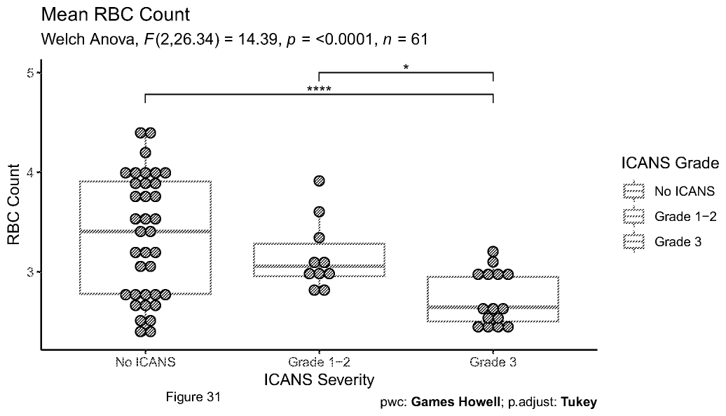

Blood count data from patient electronic health records of patients receiving CAR-T cell therapies was analyzed, and the mean red blood cell count from the first three days following CAR-T cell infusion was calculated. As shown in FIG. 31, patients that went on to develop Grade 3 ICANS had significantly lower red blood cell counts than those who developed no ICANS or less severe (Grade 1-2) ICANS. The asterisks represent p<0.0001 and p<0.05 respectively.

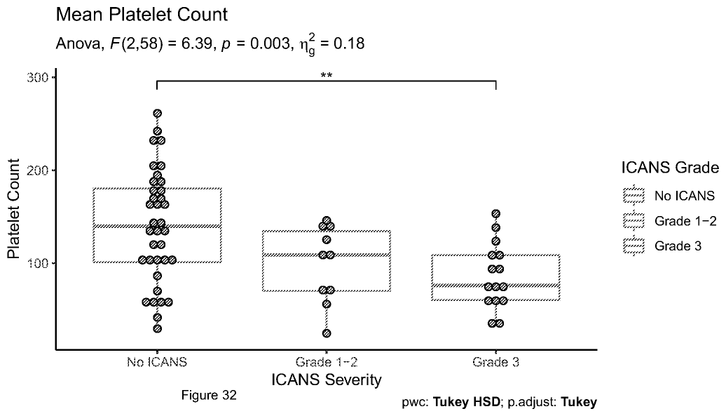

Platelet count data from patient electronic health records of patients receiving CAR-T cell therapies was analyzed, and the mean platelet count from the first three days following CAR-T cell infusion was calculated. As shown in FIG. 32, patients that went on to develop Grade 3 ICANS had significantly lower platelet counts than those that did not develop ICANS. The asterisk represents p<0.01.

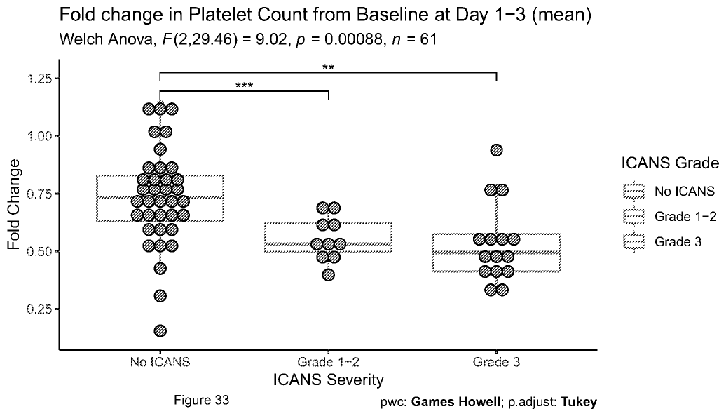

Platelet count data from patient electronic health records of patients receiving CAR-T cell therapies, and calculated the fold change in platelet count from pre-treatment baseline to first three days following CAR-T cell infusion. As shown in FIG. 33, patients that went on to develop Grade 1-2 or Grade 3 ICANS had significantly greater fold decrease from baseline than those that did not develop ICANS. The asterisks represent p<0.01 and p<0.001 respectively.

### Example 5—Combination of a Blood Biomarkers Predictive of ICANS

The following analyses was conducted on the same group of patients identified in the Materials and Methods section above.

Machine learning models for the development of ICANS were created using complete blood count data from patients over the first three days following CAR-T cell infusion. Feature selection was performed using the blood count data averaged by participant from the first three days following infusion. Feature selection was performed using the Boruta package and Recursive Feature Elimination from the caret package in R Studio. This indicated to us that the most important predictors at that time period were the combination of a lymphocyte measure, a monocyte measure, neutrophil percentage, platelet count, and red blood cell count.

For training and testing of the final models, data from each timepoint was randomly split 70/30 into a training and test set, with the outcome (Grade 2-3 ICANS) balanced equally throughout. Median impute preprocessing for missing values was used with training data only. The caret package in R was used for training and tuning of the machine learning model, using the model Regularized Logistic Regression from the LiblineaR package in R. The hyperparameters used were selected automatically using the “oneSE” method for selection of the optimal model. In the training of the machine learning models, 5 fold cv was repeated 100 times with upsampling to correct for class imbalance. Data was centered and scaled. The selected model was then applied to the test set with unseen data.

Table 1 below describes results from the 6 most robust models identified, and includes the time range of data used (Column titled “Time range,” where Day 0 is day of CAR-T cells infusion mean values used) and the performance outcomes (e.g., AUC ROC represents Area Under the Curve for the Receiver Operating Characteristic curve, and sensitivity and specificity are provided for both the Training Set and for the Test Set used) when predicting Grade 2-3 ICANS in the training and test sets.

TABLE 1
Best blood count logistic regression model for ICANS prediction.

| | Time | AUC of | Training Set | Training Set | Test Set | Test Set | | Test Set | |
|---|---|---|---|---|---|---|---|---|---|
| Model Elements | range, | ROC | Sensitivity | Specificity | Accuracy | Sensitivity | | Specificity | |
| Lymphocyte Count + Monocyte | Day 1-2 | 0.82 | 72% | 86% | 93% | 83% | (5/6) | 100% | (9/9) |
| Count + RBC Count + Neutrophil | | | | | | | | | |
| Percentage + Platelet Count | | | | | | | | | |
| Lymphocyte Count + Monocyte | Day 2-3 | 0.94 | 84% | 86% | 86% | 100% | (6/6) | 75% | (6/8) |
| Count + RBC Count + Neutrophil | | | | | | | | | |
| Percentage + Platelet Count | | | | | | | | | |
| Lymphocyte Count + Monocyte | Day 1-3 | 0.89 | 75% | 87% | 87% | 100% | (6/6) | 78% | (7/9) |
| Count + RBC Count + Neutrophil | | | | | | | | | |
| Percentage + Platelet Count | | | | | | | | | |
| Lymphocyte Percentage + | Day 1-2 | 0.83 | 69% | 86% | 100% | 100% | (6/6) | 100% | (9/9) |
| Monocyte Count + Platelet Count | | | | | | | | | |
| Lymphocyte Percentage + | Day 2-3 | 0.96 | 85% | 85% | 79% | 83% | (5/6) | 75% | (6/8) |
| Monocyte Count + Platelet Count | | | | | | | | | |
| Lymphocyte Percentage + | Day 1-3 | 0.88 | 76% | 82% | 87% | 100% | (6/6) | 78% | (7/9) |
| Monocyte Count + Platelet Count | | | | | | | | | |

Modeling Details: Regularized Logistic Regression model with hyperparameters
cost = 0.5, loss = L2_dual, epsilon = 0.001

### Example 5—Further Analysis of Protein Biomarkers Predictive of ICANS

The following analyses was conducted on the same group of patients identified in the Materials and Methods section above.

Data from proteomics analysis of plasma samples from Day 0 through Day 11 of patients getting CAR-T cell therapy was analyzed to determine which proteins show different changes in abundance over time leading up to the onset of CRS, when compared to samples from participants that did not experience CRS. A linear mixed model was created, incorporating time before CRS onset, participant ID, CRS incidence, and the interaction of time and incidence. Proteins with a significant interaction term (p<0.05) and a non-significant CRS incidence term were identified to determine which changed significantly over time compared to those from control samples taken from participants that did not develop CRS.

Those proteins that changed significantly are listed in Table 2 below, along with the difference in the coefficients (“CoefDiff”; difference between the interaction term and incidence term) and significance ratio (“SigRatio”), which is the ratio of the p values of the incidence term over the interaction term. The coefficient of difference shows whether there was a positive or negative change over time in the group that developed CRS, with a positive coefficient corresponding to an increase leading up to onset. The larger the significance ratio, the more the change over time differs as a result of group (CRS incidence), or in other words the more the change over time differs between patients developing CRS vs. those not developing CRS.

TABLE 2
Proteins show different changes in abundance
over time leading up to the onset of CRS.

| Protein | CoefDiff | SigRatio |
|---|---|---|
| IFN.gamma | 0.392263 | 34.12298 |
| CXCL11 | 0.208671 | 9.864674 |
| AXIN1 | 0.183564 | 2.292797 |
| DCTN1 | 0.177054 | 4.58965 |
| CXCL9 | 0.169687 | 4.864279 |
| DEFA1 | 0.127913 | 4.809158 |
| CCL18 | 0.109922 | 7.529091 |
| IL.24 | 0.099712 | 19.08657 |
| MPO | 0.092965 | 2166.762 |
| RETN | 0.089238 | 10.15605 |
| SCF | 0.07033 | 231.4223 |
| KIT | 0.055489 | 8.481739 |
| AOC3 | 0.047046 | 3.749277 |
| DCBLD2 | −0.05033 | 128.7906 |
| CD6 | −0.05954 | 51.87178 |
| CXADR | −0.06025 | 104.7175 |
| PSG1 | −0.07951 | 15.59889 |
| TNFRSF13C | −0.08625 | 28.36507 |
| ADA | −0.08928 | 80.72619 |
| CPB1 | −0.09024 | 9.75066 |
| CPA1 | −0.0958 | 15.95677 |
| HMOX2 | −0.12473 | 50.29339 |

Thus, the proteins in Table 2 represent predictive biomarkers of CRS that are based on the fact that their direction and/or magnitude of change over time is different between patients who develop CRS vs. those who do not.

Next, data from proteomics analysis of plasma samples from Day 0 through Day 11 of patients getting CAR-T cell therapy was analyzed to determine which proteins are differentially abundant during the time period leading to onset of CRS, when compared to samples from participants that did not experience CRS. A linear mixed model was created, incorporating time before CRS onset, participant ID, CRS incidence, and the interaction of time and incidence. Proteins with a significant CRS incidence term were identified to determine which were constitutively elevated or lower during the designated time period compared to control samples taken from participants that did not develop CRS.

Those proteins that changed significantly are listed in Table 3 along with the p value and coefficient of the CRS incidence term, which signals whether the protein was elevated or lower (i.e., positive or negative values) and to what magnitude, in patients who developed CRS.

TABLE 3
Proteins which are differentially abundant during
the time period leading to onset of CRS.

| Protein | Predictor | Coefficient | P_Value |
|---|---|---|---|
| IL7 | CRS | 1.258054 | 0.000445 |
| SIT1 | CRS | −1.79019 | 0.000472 |
| FCRL6 | CRS | −1.44948 | 0.002903 |
| IL15 | CRS | 1.63017 | 0.003911 |
| CD33 | CRS | 1.647371 | 0.006282 |
| CXCL16 | CRS | 1.235505 | 0.006543 |
| PLXNA4 | CRS | 0.34314 | 0.009424 |
| ST1A1 | CRS | 0.357166 | 0.017165 |
| CLSTN1 | CRS | 0.165372 | 0.019477 |
| PAI | CRS | 1.189783 | 0.020933 |
| TIMP4 | CRS | −0.56429 | 0.021145 |
| SPRY2 | CRS | −0.65217 | 0.023785 |
| CCL24 | CRS | −1.42085 | 0.02447 |
| CSF.1 | CRS | 0.272436 | 0.030207 |
| IL.10RA | CRS | −0.37679 | 0.033943 |
| IL.1.alpha | CRS | 0.200624 | 0.035866 |
| DPEP1 | CRS | −0.61029 | 0.036308 |
| CXCL12 | CRS | 0.2056 | 0.040399 |
| NF2 | CRS | 0.294411 | 0.041073 |
| SH2D1A | CRS | −1.11126 | 0.043611 |
| CFHR5 | CRS | 0.562078 | 0.047566 |

Thus, the proteins in Table 3 represent proteins that show a difference in abundance in plasma in patients who subsequently develop CRS vs. those who do not, without showing a difference in the change over time between the two groups of patients.

Data from proteomics analysis of plasma samples from Day 0 through Day 11 of patients getting CAR-T cell therapy was analyzed to determine which proteins show different changes in abundance over time leading to onset of ICANS, when compared to samples from participants that did not experience ICANS. A linear mixed model was created, incorporating time before ICANS onset, participant ID, ICANS incidence, and the interaction of time and incidence. Proteins with a significant interaction term (p<0.05) and a non-significant ICANS incidence term were identified to determine which changed significantly over time compared to those from control samples taken from participants that did not develop ICANS.

Those proteins that changed significantly are listed in Table 4, along with the difference in the coefficients (“CoefDiff”; difference between the interaction term and incidence term) and significance ratio (“SigRatio”), which is the ratio of the p values of the incidence term over the interaction term. The coefficient of difference shows whether there was a positive or negative change over time in the group that developed ICANS, with a positive coefficient corresponding to an increase leading up to onset. The larger the significance ratio, the more the change over time differs as a result of group (ICANS incidence), or in other words the more the change over time differs between patients developing ICANS vs. those not developing ICANS.

TABLE 4
Proteins show different changes in abundance
over time leading up to the onset of ICANS.

| Protein | CoefDiff | SigRatio |
|---|---|---|
| SH2D1A | 0.149459 | 255.9327 |
| REG1A | 0.144549 | 17.21389 |
| TIMD4 | 0.133295 | 1273513 |
| MB | 0.125292 | 2312.585 |
| KIR2DL3 | 0.125258 | 447862.3 |
| CD28 | 0.112861 | 34881.23 |
| CD8A | 0.106673 | 105.4192 |
| ST2 | 0.104263 | 20.19161 |
| IL.6RA | 0.101144 | 123.8936 |
| IL18 | 0.100896 | 1119.817 |
| CXCL1 | 0.100891 | 103.7181 |
| LTBP3 | 0.083806 | 2.512353 |
| MCP.3 | 0.083593 | 37.89921 |
| MPO | 0.074541 | 1022.589 |
| IL2.RA | 0.067992 | 39.82052 |
| CD163 | 0.064259 | 17.99246 |
| IFNL1 | 0.063875 | 31.495 |
| Notch.3 | 0.058104 | 3.102738 |
| CA3 | 0.051927 | 29.83955 |
| AP.N | 0.051027 | 5.129309 |
| IL8 | 0.041086 | 2.206221 |
| IL12RB1 | 0.040318 | 22.05697 |
| U.PAR | 0.032713 | 21.75258 |
| GPNMB | −0.01621 | 18.53678 |
| Gal.3 | −0.01995 | 5.29252 |
| PAM | −0.02124 | 14.63045 |
| LY75 | −0.02421 | 13.01663 |
| SELL | −0.02797 | 10.99935 |
| SLAMF1 | −0.03265 | 13.88597 |
| IL15 | −0.03428 | 3.016537 |
| TNFRSF9 | −0.03604 | 5.704718 |
| IGFBP6 | −0.03639 | 66.31797 |
| APOM | −0.04382 | 219.6817 |
| CTSD | −0.044 | 15.99489 |
| MASP1 | −0.05115 | 724.0592 |
| PSG1 | −0.05161 | 54.25464 |
| CHIT1 | −0.05185 | 21.70862 |
| ANGPTL3 | −0.05507 | 60.35619 |
| GP1BA | −0.05709 | 134.9444 |
| EN.RAGE | −0.05988 | 669.2801 |
| PPP3R1 | −0.06 | 71.23415 |
| CD83 | −0.06064 | 528.7509 |
| PRDX3 | −0.06203 | 18.40204 |
| IGFBP3 | −0.06388 | 220.4722 |
| ECE1 | −0.0667 | 21.55769 |
| CLEC4A | −0.07101 | 138.3847 |
| CASP.8 | −0.07231 | 167.9857 |
| QPCT | −0.07337 | 18975.46 |
| MAD1L1 | −0.07421 | 100.9989 |
| SOD1 | −0.07446 | 1.384695 |
| C1QTNF1 | −0.07937 | 40.26201 |
| SRPK2 | −0.08073 | 5.889213 |
| FAM3B | −0.08335 | 1475.852 |
| TRAF2 | −0.08399 | 32.08299 |
| SCF | −0.08874 | 184.6199 |
| CD6 | −0.09335 | 68822.41 |
| STAMBP | −0.094 | 189.6367 |
| CR2 | −0.09848 | 11543609 |
| PSME1 | −0.0985 | 12.77244 |
| SAA4 | −0.1055 | 39.64937 |
| PSIP1 | −0.10997 | 8655.945 |
| TANK | −0.11395 | 2.194742 |
| COMP | −0.12081 | 25.54469 |
| ADA | −0.12278 | 676811.1 |
| AKT1S1 | −0.13876 | 66.45469 |
| HEXIM1 | −0.14866 | 59.00966 |
| ANXA10 | −0.14928 | 62.28265 |
| HMOX2 | −0.16449 | 78727.47 |
| HCLS1 | −0.18293 | 75.76903 |
| TNFRSF13C | −0.18761 | 1.69E+10 |
| X4E.BP1 | −0.18994 | 50.16284 |

Thus, the proteins in Table 4 represent predictive biomarkers of ICANS that are based on the fact that their direction and/or magnitude of change over time is different between patients who develop ICANS vs. those who do not.

Data from proteomics analysis of plasma samples from Day 0 through Day 11 of patients getting CAR-T cell therapy was analyzed to determine which proteins are differentially abundant during the time period leading to onset of ICANS, when compared to samples from participants that did not experience ICANS. A linear mixed model was created, incorporating time before ICANS onset, participant ID, ICANS incidence, and the interaction of time and incidence. Proteins with a significant ICANS incidence term were identified to determine which were constitutively elevated or lower during the designated time period compared to control samples taken from participants that did not develop ICANS.

Those proteins that changed significantly are listed in the Table along with the p value and coefficient of the ICANS incidence term, which signals whether the protein was elevated or lower (i.e., positive or negative values) and to what magnitude, in patients who developed ICANS.

TABLE 5
Proteins that are differentially abundant during
the time period leading to onset of ICANS.

| Protein | Predictor | Coefficient | P_Value |
|---|---|---|---|
| TIMD4 | NT (ICANS) | 1.835136 | 1.74E−07 |
| TNXB | NT (ICANS) | −0.31158 | 0.000329 |
| CXCL1 | NT (ICANS) | 1.304552 | 0.000541 |
| DPEP1 | NT (ICANS) | −0.64499 | 0.001157 |
| TRAF2 | NT (ICANS) | −0.70722 | 0.001381 |
| IGFBP.2 | NT (ICANS) | 1.130477 | 0.001571 |
| IL8 | NT (ICANS) | 1.598347 | 0.001741 |
| CCL18 | NT (ICANS) | 0.975377 | 0.001798 |
| TANK | NT (ICANS) | −0.55923 | 0.002543 |
| ITGA11 | NT (ICANS) | −0.39784 | 0.00257 |
| ASGR1 | NT (ICANS) | 0.777856 | 0.004076 |
| CNTNAP2 | NT (ICANS) | −0.41029 | 0.005176 |
| CX3CL1 | NT (ICANS) | 0.809952 | 0.006752 |
| ITGB2 | NT (ICANS) | −0.81716 | 0.00701 |
| TR.AP | NT (ICANS) | 0.647408 | 0.008139 |
| FUT8 | NT (ICANS) | −0.49861 | 0.008193 |
| CLEC4A | NT (ICANS) | −0.54061 | 0.008419 |
| MCP.4 | NT (ICANS) | 1.310631 | 0.008437 |
| PROC | NT (ICANS) | −0.42023 | 0.009639 |
| IL6 | NT (ICANS) | 2.000477 | 0.011415 |
| IL6.1 | NT (ICANS) | 1.86571 | 0.013913 |
| LYVE1 | NT (ICANS) | 0.610201 | 0.014661 |
| CR2 | NT (ICANS) | −0.95866 | 0.015004 |
| TIE1 | NT (ICANS) | −0.26166 | 0.016543 |
| IFN.gamma | NT (ICANS) | 2.080102 | 0.018358 |
| MMP.9 | NT (ICANS) | −0.78536 | 0.018933 |
| PAM | NT (ICANS) | −0.32671 | 0.019996 |
| CASP.3 | NT (ICANS) | −0.63536 | 0.020376 |
| SLAMF1 | NT (ICANS) | −0.5692 | 0.020746 |
| IL.17C | NT (ICANS) | 1.000811 | 0.02091 |
| CD244 | NT (ICANS) | −0.5138 | 0.022256 |
| Gal.3 | NT (ICANS) | −0.43035 | 0.02264 |
| SMOC1 | NT (ICANS) | 0.798325 | 0.023244 |
| ISLR2 | NT (ICANS) | −0.26713 | 0.023296 |
| EFEMP1 | NT (ICANS) | 0.569551 | 0.024189 |
| EIF4B | NT (ICANS) | −0.96578 | 0.024992 |
| BACH1 | NT (ICANS) | −0.41898 | 0.02622 |
| KIR2DL3 | NT (ICANS) | 0.78999 | 0.026332 |
| CDH17 | NT (ICANS) | −1.06015 | 0.026816 |
| HGF | NT (ICANS) | 0.968365 | 0.027712 |
| IGFBP3 | NT (ICANS) | −0.7825 | 0.02796 |
| CD6 | NT (ICANS) | −1.14115 | 0.028076 |
| PIK3AP1 | NT (ICANS) | −0.62173 | 0.028378 |
| ST2 | NT (ICANS) | 1.135997 | 0.02916 |
| IL15 | NT (ICANS) | 0.950838 | 0.029208 |
| uPA.1 | NT (ICANS) | −0.49373 | 0.030739 |
| IL2.RA | NT (ICANS) | 1.315701 | 0.032709 |
| AXIN1 | NT (ICANS) | −0.46406 | 0.032876 |
| QPCT | NT (ICANS) | −0.31788 | 0.033004 |
| AKT1S1 | NT (ICANS) | −0.58976 | 0.035147 |
| REG1A | NT (ICANS) | 0.733564 | 0.036067 |
| TDGF1 | NT (ICANS) | −2.04701 | 0.036176 |
| DNER | NT (ICANS) | −0.25967 | 0.036279 |
| DAPP1 | NT (ICANS) | −0.4083 | 0.036988 |
| COMP | NT (ICANS) | −0.94095 | 0.037116 |
| IL33 | NT (ICANS) | 0.178609 | 0.037803 |
| IGFBP.1 | NT (ICANS) | 0.910658 | 0.038784 |
| RBKS | NT (ICANS) | −0.71166 | 0.040626 |
| FGF.21 | NT (ICANS) | 1.515132 | 0.044692 |
| PGLYRP1 | NT (ICANS) | −1.29599 | 0.044928 |
| Flt3L | NT (ICANS) | 0.668327 | 0.045515 |
| IL18 | NT (ICANS) | 0.958149 | 0.045534 |
| Notch.3 | NT (ICANS) | 0.381646 | 0.045811 |
| CRADD | NT (ICANS) | −0.4954 | 0.046032 |
| MET | NT (ICANS) | 0.194653 | 0.048573 |
| AARSD1 | NT (ICANS) | −0.58158 | 0.048753 |
| SPRY2 | NT (ICANS) | −0.42337 | 0.049257 |
| LTBP3 | NT (ICANS) | 0.553795 | 0.049512 |

Thus, the proteins in this Table represent proteins that show a difference in abundance in plasma in patients who subsequently develop ICANS vs. those who do not, without showing a difference in the change over time between the two groups of patients.

What is claimed is:

1. A method of treating a subject comprising:
(a) determining that the subject has an elevated level of
(i) calprotectin or citrullinated histone H3 (CitH3) in a plasma sample, or
(ii) neutrophil count, or neutrophil:lymphocyte ratio; and
(b) administering an immunotherapy to the subject.

2. The method of claim 1, further comprising administering an anti-cytokine release syndrome (anti-CRS) therapy to the subject,

3. The method of claim 2, wherein the anti-CRS therapy is administered to the subject before the immunotherapy.

4. The method of claim 3, wherein the anti-CRS therapy is administered to the subject 1-5 days before administration of the immunotherapy.

5. The method of claim 2, wherein the anti-CRS therapy is administered to the subject after the immunotherapy.

6. The method of claim 5, comprising administering the anti-CRS therapy to the subject within 5 hours after administration of the immunotherapy.

7. The method of claim 5, comprising administering the anti-CRS therapy to the subject within 1-3 hours after administration of the immunotherapy.

8. The method of claim 2, comprising administering the anti-CRS therapy to the subject concurrently with the immunotherapy.

9. The method of any one of claims 1-8, wherein the method comprises determining the level of calprotectin, CitH3, and/or neutrophil count in a plasma sample from a subject before administering the immunotherapy to the subject.

10. The method of claim 9, wherein the determining step is performed between 1-7 days prior to administering the immunotherapy.

11. The method of any one of claims 1-10, wherein the elevated level of calprotectin in the sample comprises an amount that is above a threshold that is set in the range of 1-4 standard deviations above the mean value in a reference control group of patients that did not experience CRS, and/or in a reference control group that only developed mild (Grade 1) CRS).

12. The method of any one of claims 1-11, wherein the elevated level of calprotectin in the sample comprises an amount that is ≥1,000 ng/ml.

13. The method of any one of claims 1-12, wherein the elevated level of CitH3 in the sample comprises an amount that is that is above a threshold that is set in the range of 1-4 standard deviations above the mean value in a reference control group of patients that did not experience CRS, and/or in a reference control group that developed only mild CRS (grade 1).

14. The method of any one of claims 1-13, wherein the elevated level of CitH3 in the sample comprises an amount that is that is ≥5 ng/ml.

15. The method of any one of claims 1-14, comprising monitoring the body temperature of the subject before administering the anti-CRS therapy to the subject.

16. The method of claim 15, wherein the monitoring step comprises continuous temperature monitoring of the subject.

17. The method of claim 15 or claim 16, comprising administering an anti-CRS therapy to the subject when the body temperature of the subject is elevated compared to baseline.

18. The method of any one of claims 1-17, wherein the anti-CRS therapy comprises administering an anti-IL-1 agent, an anti-IL-6 agent, an anti-IL-6R agent, or an anti-TNF agent to the subject.

19. The method of any one of claims 1-18, wherein the anti-CRS therapy comprises administering tocilizumab to the subject.

20. The method of any one of claims 1-19, wherein the anti-CRS therapy comprises administering vasoactive compounds, corticosteroids, or mechanical ventilation to the patient.

21. The method of any one of claims 1-20, wherein the elevated level of neutrophil count in the sample comprises at least 1,000 cells/μL.

22. The method of any one of claims 1-21, wherein the baseline blood neutrophil count:lymphocyte count ratio is above a threshold set in the range of 1-4 standard deviations above the mean value of this ratio in a reference control group of patients that did not experience CRS, and/or in a reference control group that developed only mild CRS (grade 1).

23. The method of any one of claims 1-21, wherein the baseline blood neutrophil count:lymphocyte count ratio that is at least 20% above the mean value of this ratio in a reference control group of patients that did not experience CRS, and/or in a reference control group that developed only mild CRS (grade 1).

24. The method of any one of claims 1-23, wherein the baseline blood neutrophil count:lymphocyte count ratio that is above 2.

25. The method of any one of claims 1-24, wherein the immunotherapy is CAR T cell therapy.

26. The method of any one of claims 1-24, the immunotherapy is an antibody.

27. The method of claim 26, wherein the antibody is an anti-CD3 antibody (OKT3), an anti-CD2 antibody (LO-CD2a), an anti-CD20 antibody (rituximab, tositumomab and |131-tositumomab), an anti-CD28 antibody (TGN1412), an anti-CD52 antibody (alemtuzumab), a CD40 agonist antibody (CP-870,893), a CD3/CD19 bispecific antibody (blinatumomab), an anti-PD-1 antibody (nivolumab), or an anti-IL-2R antibody (basiliximab and daclizumab).

28. The method of any one of claims 1-27, wherein the subject is suffering from cancer.

29. The method of claim 28, wherein the cancer is acute leukemia, lymphoma, or multiple myeloma.

30. A method of treating a subject comprising:
(a) determining that the subject has an elevated level of a protein biomarker of neurotoxicity or of neurotoxicity risk in a plasma sample prior to receiving an immunotherapy, wherein the protein biomarker is calprotectin is calprotectin, Secreted Frizzled Related Protein 1 (SFRP1), hepatocyte growth factor (HGF), secreted modular calcium-binding protein 1 (SMOC1), 6-pyruvoyl tetrahydrobiopterin synthase (PTS), low-density lipoprotein (LDL) receptor, tissue plasminogen activator (tPA), tumor necrosis factor-like weak inducer of apoptosis (TWEAK), C-C Motif Chemokine Ligand 18 (CCL18), von Willebrand Factor (vWF), Thrombospondin-related anonymous protein (TRAP), stem cell factor (SCF), Insulin Like Growth Factor Binding Protein 3 (IGFBP3) or Defensin Beta 4A (DEFB4A); and
(b) administering an immunotherapy to the subject.

31. The method of claim 30, further comprising administering an anti-neurotoxicity therapy to the subject.

32. The method of claim 31, wherein the anti-neurotoxicity therapy is administered to the subject before the immunotherapy.

33. The method of claim 32, wherein the anti-neurotoxicity therapy is administered to the subject 1-5 days before administration of the immunotherapy.

34. The method of claim 31, wherein the anti-neurotoxicity therapy is administered to the subject after the immunotherapy.

35. The method of claim 34, comprising administering the anti-neurotoxicity therapy to the subject within 5 hours after administration of the immunotherapy.

36. The method of claim 34, comprising administering the anti-neurotoxicity therapy to the subject within 1-3 hours after administration of the immunotherapy.

37. The method of claim 31, comprising administering the anti-neurotoxicity therapy to the subject concurrently with the immunotherapy.

38. The method of any one of claims 30-37, wherein the method comprises determining the level of protein biomarker of neurotoxicity in a plasma sample from the subject before administering the immunotherapy to the subject.

39. The method of any one of claims 30-38, wherein method comprises determining the level of calprotectin in a plasma sample from the subject before administering the CAR-T cell therapy to the subject.

40. The method of any one of claims 30-39, wherein the determining step is performed between 1-7 days prior to administering the immunotherapy.

41. The method of claim 39, wherein the elevated level of calprotectin in the sample comprises ≥1,000 ng/mL.

42. The method of claim 39, wherein the elevated level of calprotectin in the sample comprises an amount that is above a threshold that is set in the range of 1-4 standard deviations above the mean value in a reference control group of patients that did not experience neurotoxicity, and/or in a reference control group that only developed mild neurotoxicity.

43. A method of treating a subject comprising:
(a) administering an immunotherapy to the subject; and
(b) determining that the subject has an elevated level of TIMD4 compared to baseline, or an elevated rate of change in TIMD4 in a plasma sample after receiving the immunotherapy.

44. The method of claim 43, wherein the anti-neurotoxicity therapy is administered to the subject before the immunotherapy.

45. The method of claim 44, wherein the anti-neurotoxicity therapy is administered to the subject 1-5 days before administration of the immunotherapy.

46. The method of claim 43, wherein the anti-neurotoxicity therapy is administered to the subject after the immunotherapy.

47. The method of claim 46, comprising administering the anti-neurotoxicity therapy to the subject within 5 hours after administration of the immunotherapy.

48. The method of claim 46, comprising administering the anti-neurotoxicity therapy to the subject within 1-3 hours after administration of the immunotherapy.

49. The method of claim 43, comprising administering the anti-neurotoxicity therapy to the subject concurrently with the immunotherapy.

50. The method of claim 43, wherein method comprises determining the level of TIMD4 in a plasma sample from the subject after administering the immunotherapy to the subject.

51. The method of claim 50, wherein the elevated level of TIMD4 in the sample comprises an amount that is ≥10% increase compared to baseline.

52. The method of claim 43 or claim 44, wherein the determining step is performed between 1-7 days after administering the immunotherapy.

53. The method of claim 43, wherein the method comprises determining the elevated rate of change in TIMD4 level in a plasma sample from the subject before and after administering the immunotherapy to the subject.

53. A method of treating a subject comprising
(a) administering an immunotherapy to the subject;
(b) determining that the subject has an elevated level, or elevated rate of increase, of one or more of CXCL1, IGFBP, IL-8, CCL18, ASGR1, CX3CL1, TRAP, MCP, IL-6, IL-16, LYVE1, IFNγ, IL-17, SMOC1, EFEMP1, KIR2DL3, HGF, ST2, IL-15, IL2RA, REG1A, IL-33, IGFBP1, FGF21, FIt3L, IL-18, Notch3, MET, and LTBP3 in a sample after receiving said immunotherapy; and
(c) administering an anti-neurotoxicity agent to the subject.

54. A method of treating a subject comprising:
(a) determining that the subject has an elevated level of monocyte/lymphocyte ratio in a plasma sample; and
(b) administering an immunotherapy to the subject.

55. The method of claim 55, further comprising administering an anti-cytokine release syndrome (anti-CRS) therapy to the subject,

56. The method of claim 55, wherein the anti-CRS therapy is administered to the subject before the immunotherapy.

57. The method of claim 56, wherein the anti-CRS therapy is administered to the subject 1-5 days before administration of the immunotherapy.

58. The method of claim 55, wherein the anti-CRS therapy is administered to the subject after the immunotherapy.

59. A method of treating a subject comprising:
(a) administering an immunotherapy to the subject;
(b) determining that the subject has an elevated level of:
(i) neutrophil/lymphocyte ratio values in a plasma sample from the subject; and/or
(ii) neutrophil percentages in a plasma sample from the subject; and
(b) administering an anti-neurotoxicity agent to the subject.

60. A method of treating a subject comprising
(a) administering an immunotherapy to the subject,
(a) determining a decreased level of one or more of lymphocyte count, monocyte count, neutrophil percentage, platelet count, and red blood cell count in a blood sample from the subject, and
(c) administering an anti-neurotoxicity agent or an anti-cytokine release syndrome (anti-CRS) therapy to the subject.
